(19) United States
(12) Patent Application Publication
(10) Pub. No.: US 2024/0207268 A1
(43) Pub. Date: Jun. 27, 2024

(54) PYRAZOLYLPYRIMIDINES FOR TREATING MALIGNANT SOLID TUMOR

(71) Applicant: BIOTHERYX, INC., San Diego, CA (US)

(72) Inventors: Kyle W.H. CHAN, San Diego, CA (US); Aparajita Hoskote CHOURASIA, San Diego, CA (US); Dung Zung THAI, Redwood City, CA (US)

(21) Appl. No.: 18/551,926

(22) PCT Filed: Mar. 23, 2022
(86) PCT No.: PCT/US2022/021447

§ 371 Date: Sep. 22, 2023

Related U.S. Application Data
(60) Provisional application No. 63/165,137, filed on Mar. 24, 2021.

(51) Int. Cl. A61K 31/506; A61K 31/5377

(52) U.S. Cl. CPC A61K 31/506; A61K 31/5377

(57) ABSTRACT

Provided herein is a method of treating, preventing, or ameliorating one or more symptoms of a malignant solid tumor with a pyrazolylpyrimidine, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

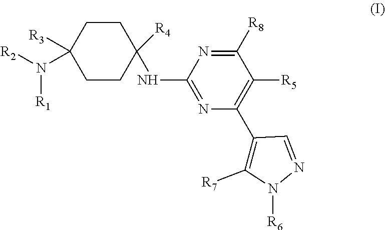

### CROSS REFERENCE TO RELATED APPLICATION

This application claims the benefit of the priority of U.S. Provisional Application No. 63/165,137, filed Mar. 24, 2021; the disclosure of which is incorporated herein by reference in its entirety.

### FIELD

Provided herein is a method of treating, preventing, or ameliorating one or more symptoms of a malignant solid tumor with a pyrazolylpyrimidine, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

### BACKGROUND

Casein kinases are serine/threonine kinases that phosphorylate proteins to mediate normal biological functions and malignant transformation. Schittek et al., Mol. Cancer 2014, 13, 231-45. Casein kinase 1 alpha (CK1α) functions as a tumor inducer in several cancers through negative regulation of Wnt/β-catenin signaling and p53. Ebert & Krönke, N. Engl. J. Med. 2018, 379, 1873-74. CK1α phosphorylates β-catenin at serine 45, leading to ubiquitination and degradation of β-catenin. Schittek et al., Mol. Cancer 2014, 13, 231-45; Ebert & Krönke, N. Engl. J. Med. 2018, 379, 1873-4; Elyada et al., Nature 2011, 470, 409-13. CK1α also phosphorylates murine double minute X (MDMX) at serine 289, resulting in enhanced binding of MDMX to p53. Wu et al., Mol. Cell. Biol. 2012, 32, 4821-32. Additionally, a complex of CK1α and mouse double minute 2 homolog (MDM2) inhibits p53. Elyada et al., Nature 2011, 470, 409-13. Thus, enhanced inhibition of CK1α with subsequent p53 activation has the potential to be effective in treating a wide array of cancers.

Despite the advances in cancer treatment, cancer (e.g., malignant solid tumor) remains a major worldwide public health problem. It was estimated that there will be 1,898,160 new cancer cases diagnosed and 608,570 cancer deaths in the US alone in 2021. Cancer Facts & Figures 2021. Therefore, there is a need for an effective therapy for cancer treatment.

### SUMMARY OF THE DISCLOSURE

Provided herein is a method of treating, preventing, or ameliorating one or more symptoms of a malignant solid tumor in a subject, comprising administering to the subject in need thereof a therapeutically effective amount of a compound of Formula (I),

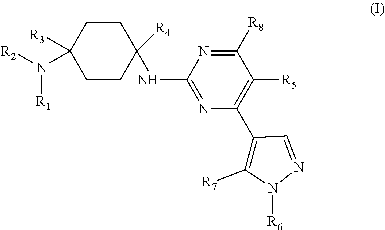

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R₁, R₂, R₃, and R₄ are (a) or (b):
   (a) R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or
      R₁ and R₂ together with the nitrogen atom to which they are attached form heteroaryl or heterocyclyl; and
      R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or
      R₃ and R₄ are linked together to form C₁₋₆ alkylene; or
   (b) R₁ is (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
      R₂ and R₃ together with the carbon and nitrogen atoms to which they are attached form heterocyclylene; and
      R₄ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, -OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₅, R₇, and R₈ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₆ is hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, alkenyl, alkynyl, cycloalkyl, aryl, aralkyl, heteroaryl, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, -13 NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a method of inhibiting the growth of a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Additionally, provided herein is a method of inducing apoptosis in a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, and mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human) without excessive toxicity, irritation, allergic response, immunogenicity, or other problems or complications, and commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The term “alkynyl” refers to a linear or branched monovalent hydrocarbon radical, which contains one or more, in one embodiment, one, two, three, or four, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C6-15), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenyl-propyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]-pyridinyl, furo[3,2-c]pyridinyl, furo[3,4-b]pyridinyl, and furo[3,4-c]pyridinyl), imidazopyridinyl (including all isomeric forms, e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]-thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]-pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]-pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno-[3,2-c]pyridinyl). In yet another embodiment, the heteroaryl is tricyclic. Examples of tricyclic heteroaryl groups include, but are not limited to, acridinyl, benzindolyl, carbazolyl, dibenzo-furanyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. Heterocyclylene groups are bonded to the rest of a molecule through the non-aromatic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydropyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydroindoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydrothiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen”, “halide,” or “halo” refers to fluoro, chloro, bromo, and/or iodo.

The term “optionally substituted” is intended to mean that a group or substituent, such as an alkyl, alkylene, alkenyl, alkynyl, cycloalkyl, aryl, aralkyl, heteroaryl, heterocyclyl, or heterocyclylene group, may be substituted with one or more, in one embodiment, one, two, three, or four, substituents Q, each of which is independently selected from, e.g., (a) deuterium (-D), cyano (—CN), halo, imino (═NH), nitro (—NO₂), and oxo (═O); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, NR^{a}C(O)SR^{d}, —NR^{a}C(NR^{d})NR^{b}R^{c}, —NR^{a}C(S)R^{d}, —NR^{a}C(S)OR^{d}, NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean, when referred to a substance, sufficiently homogeneous to appear free of readily detectable impurities as determined by a standard analytical method used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; or (ii) a pharmaceutically acceptable salt, solvate, hydrate, or prodrug of the compound referenced therein, or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, described herein is a compound of Formula (I),

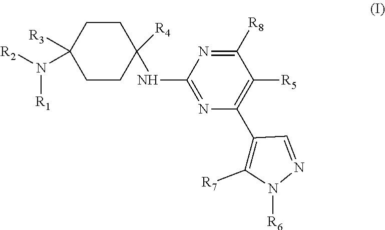

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R₁, R₂, R₃, and R₄ are (a) or (b):
   (a) R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or
      R₁ and R₂ together with the nitrogen atom to which they are attached form heteroaryl or heterocyclyl; and
      R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or
      R₃ and R₄ are linked together to form C₁₋₆ alkylene; or
   (b) R₁ is (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
      R₂ and R₃ together with the carbon and nitrogen atoms to which they are attached form heterocyclylene; and
      R₄ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₅, R₇, and R₈ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₆ is hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, alkenyl, alkynyl, cycloalkyl, aryl, aralkyl, heteroaryl, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, R₁ and R₂ are each independently (i) hydrogen; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, R₁ and R₂ are each independently —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is as defined herein. In certain embodiments, R₁ and R₂ are each independently —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, C(S)NR^{1b}R^{1c}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is as defined herein.

In certain embodiments, R₁ and R₂ are each independently (i) hydrogen; (ii) C₁₋₆ alkyl, C₂₋₆ alkynyl, C₃₋₁₀ cycloalkyl, C₆₋₁₄ aryl, or C₇₋₁₅ aralkyl, each of which is optionally substituted with one or more substituents Q; or (iii) —C(O)R^{1a}, where R^{1a} is as defined herein. In certain embodiments, R₁ and R₂ are each independently (i) hydrogen; (ii) C₁₋₆ alkyl, C₂₋₆ alkynyl, C₃₋₁₀ cycloalkyl, C₆₋₁₄ aryl, or C₇₋₁₅ aralkyl, each of which is optionally substituted with one or more substituents Q; or (iii) —C(O)R^{1a}, where R^{1a} is C₁₋₆ alkyl, C₂₋₆ alkynyl, or monocyclic heteroaryl, each of which is optionally substituted with one or more substituents Q. In certain embodiments, R₁ and R₂ are each independently hydrogen, methyl, pentyl, trifluoroethyl, hydroxylmethyl, methoxyethyl, aminopropyl, pyrazolylmethyl, (methyl-pyrazolyl)methyl, (pyrazolyl)ethyl, pyridinylmethyl, pentynyl, aminobicyclo[2.2.1]heptanyl, aminobicyclo-[2.2.2]octanyl, phenyl, benzyl, methoxyacetyl, pentynoyl, pyrazolylcarbonyl, cyclooctyloxycarbonylaminopropyl, or cyclooctenyloxycarbonylaminopropyl. In certain embodiments, R₁ and R₂ are each independently hydrogen, methyl, 1-pentyl, 2,2,2-trifluoroethyl, hydroxyl-methyl, 2-methoxyethyl, 3-aminopropyl, pyrazol-3-ylmethyl, pyrazol-4-ylmethyl, (1-methyl-pyrazol-4-yl)methyl, (3-methylpyrazol-4-yl)methyl, 1-(pyrazol-4-yl)ethyl, pyridin-3-yl-methyl, pent-4-yn-1-yl, 4-aminobicyclo[2.2.1]heptan-1-yl, 4-aminobicyclo[2.2.2]octan-1-yl, phenyl, benzyl, 2-methoxyacetyl, pent-4-ynoyl, pyrazol-3-ylcarbonyl, 3-cyclooctyloxy-carbonylaminopropyl, (E)-3-(cyclooct-4-en-1-yloxycarbonylamino)propyl, or (Z)-3-(cyclo-oct-4-en-1-yloxycarbonylamino)propyl.

In certain embodiments, R₁ is hydrogen or C₁₋₆ alkyl optionally substituted with one or more substituents Q. In certain embodiments, R₁ is hydrogen, methyl, pentyl, trifluoroethyl, or pyrazolylmethyl. In certain embodiments, R₁ is hydrogen, methyl, 1-pentyl, 2,2,2-trifluoroethyl, or pyrazol-4-ylmethyl.

In certain embodiments, R₂ is (i) hydrogen; (ii) C₁₋₆ alkyl, C₂₋₆ alkynyl, C₃₋₁₀ cycloalkyl, C₆₋₁₄ aryl, or C₇₋₁₅ aralkyl, each of which is optionally substituted with one or more substituents Q; or (iii) —C(O)R^{1a}, where R^{1a} is as defined herein. In certain embodiments, R₂ is (i) hydrogen; (ii) C₁₋₆ alkyl, C₂₋₆ alkynyl, C₃₋₁₀ cycloalkyl, C₆₋₁₄ aryl, or C₇₋₁₅ aralkyl, each of which is optionally substituted with one or more substituents Q; or (iii) —C(O)R^{1a}, where R^{1a} is C₁₋₆ alkyl, C₂₋₆ alkynyl, or monocyclic heteroaryl, each of which is optionally substituted with one or more substituents Q. In certain embodiments, R₂ is hydrogen, methyl, pentyl, trifluoroethyl, hydroxylmethyl, methoxyethyl, aminopropyl, pyrazolylmethyl, (methylpyrazolyl)methyl, (pyrazolyl)ethyl, pyridinylmethyl, pentynyl, aminobicyclo[2.2.1]heptanyl, aminobicyclo-[2.2.2]octanyl, phenyl, benzyl, methoxyacetyl, pentynoyl, pyrazolylcarbonyl, cyclooctyloxycarbonylaminopropyl, or cyclooctenyloxy-carbonylaminopropyl. In certain embodiments, R₂ is hydrogen, methyl, 1-pentyl, 2,2,2-trifluoroethyl, hydroxyl-methyl, 2-methoxyethyl, 3-aminopropyl, pyrazol-3-ylmethyl, pyrazol-4-ylmethyl, (1-methyl-pyrazol-4-yl)methyl, (3-methylpyrazol-4-yl)methyl, 1-(pyrazol-4-yl)ethyl, pyridin-3-yl-methyl, pent-4-yn-1-yl, 4-aminobicyclo[2.2.1]heptan-1-yl, 4-aminobicyclo[2.2.2]octan-1-yl, phenyl, benzyl, 2-methoxyacetyl, pent-4-ynoyl, pyrazol-3-ylcarbonyl, 3-cyclooctyloxy-carbonylaminopropyl, (E)-3-(cyclooct-4-en-1-yloxycarbonyl-amino)propyl, or (Z)-3-(cyclo-oct-4-en-1-yloxycarbonylamino)propyl.

In certain embodiments, R₁ is hydrogen. In certain embodiments, R₂ is hydrogen. In certain embodiments, R₁ and R₂ are each hydrogen.

In certain embodiments, R₁ and R₂ together with the nitrogen atom to which they are attached form heteroaryl or heterocyclyl, each of which is optionally substituted with one or more substituents Q. In certain embodiments, R₁ and R₂ together with the nitrogen atom to which they are attached form monocyclic heteroaryl or monocyclic heterocyclyl, each of which is optionally substituted with one or more substituents Q. In certain embodiments, R₁ and R₂ together with the nitrogen atom to which they are attached form pyrazolyl, imidazolyl, azetidinyl, pyrrolidinyl, piperidinyl, or morpholinyl, each of which is optionally substituted with one or more substituents Q. In certain embodiments, R₁ and R₂ together with the nitrogen atom to which they are attached form pyrazol-1-yl, imidazol-1-yl, azetidin-1-yl, pyrrolidin-1-yl, piperidin-1-yl, or morpholin-4-yl, each of which is optionally substituted with one or more substituents Q.

In certain embodiments, R₂ and R₃ together with the carbon and nitrogen atoms to which they are attached form heterocyclylene optionally substituted with one or more substituents Q. In certain embodiments, R₂ and R₃ together with the carbon and nitrogen atoms to which they are attached form monocyclic heterocyclylene optionally substituted with one or more substituents Q. In certain embodiments, R₂ and R₃ together with the carbon and nitrogen atoms to which they are attached form pyrrolidindiyl, imidazolidindiyl, or oxazolidindiyl, each of which is optionally substituted with one or more substituents Q. In certain embodiments, R₂ and R₃ together with the carbon and nitrogen atoms to which they are attached form oxopyrrolidindiyl, dioxoimidazolidindiyl, or oxazolidindiyl. In certain embodiments, R₂ and R₃ together with the carbon and nitrogen atoms to which they are attached form 5-oxo-pyrrolidin-2,2-diyl, 2,5-dioxo-imidazolidin-4,4-diyl, or 2-oxo-oxazolidin-4,4-diyl.

In certain embodiments, R₃ and R₄ are each independently (i) hydrogen, deuterium, cyano, halo, or nitro; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, R₃ and R₄ are each independently —C(O)R^{1a}, —C(O)OR^{1a}, —C(O)NR^{1b}R^{1c}, C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is as defined herein.

In certain embodiments, R₃ and R₄ are each independently hydrogen or C₁₋₆ alkyl optionally substituted with one or more substituents Q. In certain embodiments, R₃ and R₄ are each independently hydrogen or methyl. In certain embodiments, R₃ is hydrogen or C₁₋₆ alkyl optionally substituted with one or more substituents Q. In certain embodiments, R₃ is hydrogen or methyl. In certain embodiments, R₃ is hydrogen. In certain embodiments, R₄ is hydrogen or C₁₋₆ alkyl optionally substituted with one or more substituents Q. In certain embodiments, R₄ is hydrogen. In certain embodiments, R₃ and R₄ are each hydrogen.

In certain embodiments, R₃ and R₄ are linked together to form C₁₋₆ alkylene, optionally substituted with one or more substituents Q. In certain embodiments, R₃ and R₄ are linked together to form methanediyl or ethanediyl, each of which is optionally substituted with one or more substituents Q. In certain embodiments, R₃ and R₄ are linked together to form methanediyl or ethane-1,2-diyl.

In certain embodiments, R₅ is (i) hydrogen, deuterium, cyano, halo, or nitro; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, R₅ is —C(O)R^{1a}, —C(O)OR^{1a}, —C(O)NR^{1b}R^{1c}, C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR ^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is as defined herein.

In certain embodiments, R₅ is hydrogen, deuterium, cyano, halo, nitro, or C₁₋₆ alkyl optionally substituted with one or more substituents Q. In certain embodiments, R₅ is hydrogen, halo, or C₁₋₆ alkyl optionally substituted with one or more substituents Q. In certain embodiments, R₅ is hydrogen, fluoro, chloro, or methyl. In certain embodiments, R₅ is halo. In certain embodiments, R₅ is fluoro or chloro. In certain embodiments, R₅ is chloro.

In certain embodiments, R₆ is (i) hydrogen; or (ii) C₁₋₆ alkyl, C₃₋₁₀ cycloalkyl, or heterocyclyl, each of which is optionally substituted with one or more substituents Q. In certain embodiments, R₆ is (i) hydrogen; or (ii) C₁₋₆ alkyl, C₃₋₁₀ cycloalkyl, or monocyclic heterocyclyl, each of which is optionally substituted with one or more substituents Q. In certain embodiments, R₆ is hydrogen, methyl, isopropyl, cyclopentyl, oxetanyl, tetrafuranyl, or tetrahydropyranyl. In certain embodiments, R₆ is hydrogen, methyl, isopropyl, cyclopentyl, oxetan-3-yl, tetrafuran-3-yl, tetrahydropyran-3-yl, or tetrahydropyran-4-yl. In certain embodiments, R₆ is hydrogen. In certain embodiments, R₆ is methyl.

In certain embodiments, R₇ is (i) hydrogen, deuterium, cyano, halo, or nitro; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, R₇ is —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is as defined herein.

In certain embodiments, R₇ is C₁₋₆ alkyl optionally substituents with one or more substituents Q. In certain embodiments, R₇ is C₁₋₆ alkyl substituted with C₃₋₁₀ cycloalkyl, i.e., C₃₋₁₀ cycloalkyl—C₁₋₆ alkyl, wherein the alkyl and cycloalkyl are each optionally substituents with one or more substituents Q^{a}. In certain embodiments, R₇ is butyl, cyclopropylmethyl, methylcyclopropylmethyl, hydroxylcyclopropylmethyl, cyclobutylmethyl, or cyclopentylmethyl. In certain embodiments, R₇ is tert-butyl, cyclopropylmethyl, 1-methylcyclopropylmethyl, 1-hydroxyl-cyclopropylmethyl, cyclobutylmethyl, or cyclopentylmethyl. In certain embodiments, R₇ is cyclopropylmethyl, optionally substituents with one or more substituents Q. In certain embodiments, R₇ is cyclopropylmethyl.

In certain embodiments, R₈ is (i) hydrogen, deuterium, cyano, halo, or nitro; or (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q. In certain embodiments, Rs is —C(O)R^{1a}, —C(O)OR^{1a}, C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is as defined herein. In certain embodiments, Rs is hydrogen.

In one embodiment, in Formula (I),
R₁, R₂, R₃, and R₄ are (a) or (b):
   (a) R₁ and R₂ are each independently (i) hydrogen; (ii) C₁₋₆ alkyl, C₂₋₆ alkynyl, C₃₋₁₀ cycloalkyl, C₆₋₁₄ aryl, or C₇₋₁₅ aralkyl; or (iii) —C(O)R^{1a}; or R₁ and R₂ together with the nitrogen atom to which they are attached form heteroaryl or heterocyclyl; and
      R₃ is hydrogen or C₁₋₆ alkyl; or R₄ is hydrogen; or R₃ and R₄ are linked together to form C₁₋₆ alkylene; or
   (b) R₁ is (i) hydrogen; (ii) C₁₋₆ alkyl, C₂₋₆ alkynyl, C₃₋₁₀ cycloalkyl, C₆₋₁₄ aryl, or C₇₋₁₅ aralkyl; or (iii) —C(O)R^{1a};
      R₂ and R₃ together with the carbon and nitrogen atoms to which they are attached form heterocyclylene; and
R₄ is hydrogen;
R₅ is hydrogen, halo, or C₁₋₆ alkyl;
R₆ is (i) hydrogen; or (ii) C₁₋₆ alkyl, C₃₋₁₀ cycloalkyl, or heterocyclyl;
R₇ is C₁₋₆ alkyl; and
R₈ is hydrogen;
wherein each alkyl, alkylene, alkynyl, cycloalkyl, aryl, aralkyl, heteroaryl, heterocyclyl, or heterocyclylene is optionally substituted with one or more substituents Q.

In another embodiment, in Formula (I),
R₁, R₂, R₃, and R₄ are (a) or (b):
   (a) R₁ is hydrogen or C₁₋₆ alkyl;
      R₂ is (i) hydrogen; (ii) C₁₋₆ alkyl, C₂₋₆ alkynyl, C₃₋₁₀ cycloalkyl, C₆₋₁₄ aryl, or C₇₋₁₅ aralkyl; or (iii) —C(O)R^{1a}, where R^{1a} is C₁₋₆ alkyl, C₂₋₆ alkynyl, or heteroaryl; or R₁ and R₂ together with the nitrogen atom to which they are attached form monocyclic heteroaryl or monocyclic heterocyclyl; or
      R₂ and R₃ together with the carbon and nitrogen atoms to which they are attached form monocyclic heterocyclylene;
      R₃ is hydrogen or C₁₋₆ alkyl;
      R₄ is hydrogen; and
      R₃ and R₄ are linked together to form C₁₋₆ alkylene; or
   (b) R₁ is hydrogen or C₁₋₆ alkyl;
      R₂ and R₃ together with the carbon and nitrogen atoms to which they are attached form monocyclic heterocyclylene; and
      R₄ is hydrogen;
R₅ is hydrogen, halo, or C₁₋₆ alkyl;
R₆ is (i) hydrogen; or (ii) C₁₋₆ alkyl, C₃₋₁₀ cycloalkyl, or monocyclic heterocyclyl;
R₇ is C₁₋₆ alkyl or C₃₋₁₀ cycloalkyl-C₁₋₆ alkyl; and
R₈ is hydrogen;
wherein each alkyl, alkylene, alkynyl, cycloalkyl, aryl, aralkyl, heteroaryl, heterocyclyl, or heterocyclylene is optionally substituted with one or more substituents Q.

In yet another embodiment, in Formula (I),
R₁, R₂, R₃, and R₄ are (a) or (b):
   (a) R₁ is hydrogen, methyl, pentyl, trifluoroethyl, or pyrazolylmethyl;
      R₂ is hydrogen, methyl, pentyl, trifluoroethyl, hydroxylmethyl, methoxyethyl, aminopropyl, pyrazolylmethyl, (methylpyrazolyl)methyl, (pyrazolyl)ethyl, pyridinylmethyl, pentynyl, aminobicyclo[2.2.1]heptanyl, aminobicyclo[2.2.2]-octanyl, phenyl, benzyl, methoxyacetyl, pentynoyl, pyrazolylcarbonyl, cyclooctyl-oxycarbonylaminopropyl, or cyclooctenyloxycarbonylaminopropyl; or
      R₁ and R₂ together with the nitrogen atom to which they are attached form pyrazolyl, imidazolyl, azetidinyl, pyrrolidinyl, piperidinyl, or morpholinyl; or
      R₃ is hydrogen or methyl; and
      R₄ is hydrogen; or
      R₃ and R₄ together form methanediyl or ethanediyl; or
   (b) R₁ is hydrogen, methyl, pentyl, trifluoroethyl, or pyrazolylmethyl;
      R₂ and R₃ together with the carbon and nitrogen atoms to which they are attached form oxopyrrolidindiyl, dioxoimidazolidindiyl, or oxazolidindiyl; and
      R₄ is hydrogen;
R₅ is hydrogen, fluoro, chloro, or methyl;
R₆ is hydrogen, methyl, isopropyl, cyclopentyl, oxetanyl, tetrafuranyl, or tetrahydropyranyl;
R₇ is butyl, cyclopropylmethyl, methylcyclopropylmethyl, hydroxylcyclopropylmethyl, cyclobutylmethyl, or cyclopentylmethyl; and
R₈ is hydrogen.

In still another embodiment, in Formula (I),
R₁, R₂, R₃, and R₄ are (a) or (b):
   (a) R₁ is hydrogen, methyl, 1-pentyl, 2,2,2-trifluoroethyl, or pyrazol-4-ylmethyl;
      R₂ is hydrogen, methyl, 1-pentyl, 2,2,2-trifluoroethyl, hydroxylmethyl, 2-methoxyethyl, 3-aminopropyl, pyrazol-3-ylmethyl, pyrazol-4-ylmethyl, (1-methylpyrazol-4-yl)methyl, (3-methylpyrazol-4-yl)methyl, 1-(pyrazol-4-yl)ethyl, pyridin-3-ylmethyl, pent-4-yn-1-yl, 4-aminobicyclo[2.2.1]heptan-1-yl, 4-amino-bicyclo[2.2.2]octan-1-yl, phenyl, benzyl, 2-methoxyacetyl, pent-4-ynoyl, pyrazol-3-ylcarbonyl, 3-cyclooctyloxycarbonylaminopropyl, (E)-3-(cyclooct-4-en-1-yl-oxycarbonylamino)propyl, or (Z)-3-(cyclooct-4-en-1-yloxy-carbonylamino)-propyl; or
      R₁ and R₂ together with the nitrogen atom to which they are attached form pyrazol-1-yl, imidazol-1-yl, azetidin-1-yl, pyrrolidin-1-yl, piperidin-1-yl, or morpholin-4-yl;
      R₃ is hydrogen or methyl; and
      R₄ is hydrogen; or
      R₃ and R₄ are linked together to form methanediyl or ethane-1,2-diyl; or
   (b) R₁ is hydrogen, methyl, 1-pentyl, 2,2,2-trifluoroethyl, or pyrazol-4-ylmethyl;
      R₂ and R₃ together with the carbon and nitrogen atoms to which they are attached form 5-oxo-pyrrolidin-2,2-diyl, 2,5-dioxo-imidazolidin-4,4-diyl, or 2-oxo-oxazolidin-4,4-diyl; and
      R₄ is hydrogen;
R₅ is hydrogen, fluoro, chloro, or methyl;
R₆ is hydrogen, methyl, isopropyl, cyclopentyl, oxetan-3-yl, tetrafuran-3-yl, tetrahydropyran-3-yl, or tetrahydropyran-4-yl;
R₇ is tert-butyl, cyclopropylmethyl, 1-methylcyclopropylmethyl, 1-hydroxylcyclopropylmethyl, cyclobutylmethyl, or cyclopentylmethyl; and
R₈ is hydrogen.

In another embodiment, described herein is:

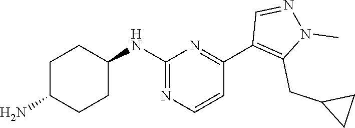 [SMILES: Cn1ncc(-c2ccnc(N[C@H]3CC[C@H](N)CC3)n2)c1CC1CC1]

(1r,4r)-N¹-(4-(5-(cyclopropylmethyl)-1-methyl-1H-pyrazol-4-yl)pyrimidin-2-yl)cyclohexane-1,4-diamine A1;

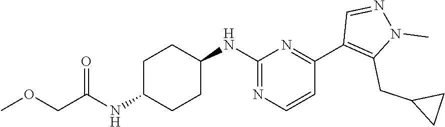 [SMILES: COCC(=O)N[C@H]1CC[C@H](Nc2nccc(-c3cnn(C)c3CC3CC3)n2)CC1]

N-((1r,4r)-4-((4-(5-(cyclopropylmethyl)-1-methyl-1H-pyrazol-4-yl)pyrimidin-2-yl)amino)cyclohexyl)-2-methoxyacetamide A2;

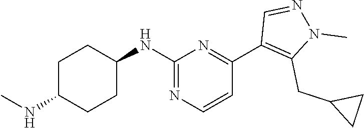 [SMILES: CN[C@H]1CC[C@H](Nc2nccc(-c3cnn(C)c3CC3CC3)n2)CC1]

(1r,4r)-N¹-(4-(5-(cyclopropylmethyl)-1-methyl-1H-pyrazol-4-yl)pyrimidin-2-yl)-N⁴-methylcyclohexane-1,4-diamine A3;

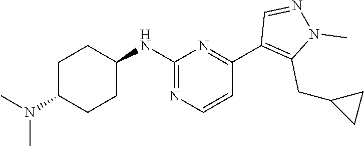 [SMILES: Cn1ncc(-c2ccnc(N[C@H]3CC[C@H](N(C)C)CC3)n2)c1CC1CC1]

(1r,4r)-N¹-(4-(5-(cyclopropylmethyl)-1-methyl-1H-pyrazol-4-yl)pyrimidin-2-yl)-N⁴,N⁴-dimethylcyclohexane-1,4-diamine A4;

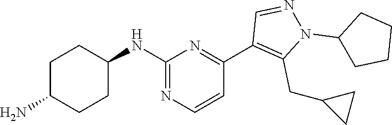 [SMILES: N[C@H]1CC[C@H](Nc2nccc(-c3cnn(C4CCCC4)c3CC3CC3)n2)CC1]

(1r,4r)-N¹-(4-(1-cyclopentyl-5-(cyclopropylmethyl)-1H-pyrazol-4-yl)pyrimidin-2-yl)cyclohexane-1,4-diamine A5;

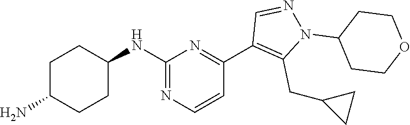 [SMILES: N[C@H]1CC[C@H](Nc2nccc(-c3cnn(C4CCOCC4)c3CC3CC3)n2)CC1]

(1r,4r)-N¹-(4-(5-(cyclopropylmethyl)-1-(tetrahydro-2H-pyran-4-yl)-1H-pyrazol-4-yl)pyrimidin-2-yl)cyclohexane-1,4-diamine A6;

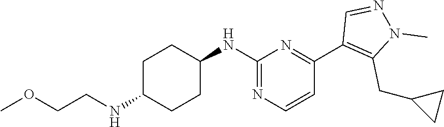 [SMILES: COCCN[C@H]1CC[C@H](Nc2nccc(-c3cnn(C)c3CC3CC3)n2)CC1]

(1r,4r)-N¹-4-(5-(cyclopropylmethyl)-1-methyl-1H-pyrazol-4-yl)pyrimidin-2-yl)-N⁴-(2-methoxyethyl)cyclohexane-1,4-diamine A7;

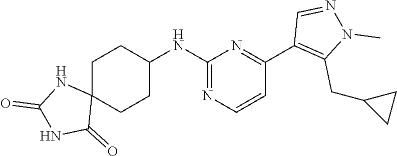 [SMILES: Cn1ncc(-c2ccnc(NC3CCC4(CC3)NC(=O)NC4=O)n2)c1CC1CC1]

8-((4-(5-(cyclopropylmethyl)-1-methyl-1H-pyrazol-4-yl)pyrimidin-2-yl)amino)-1,3-diazaspiro[4.5]decane-2,4-dione A8;

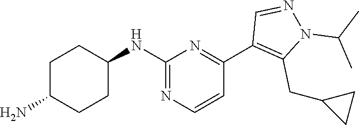 [SMILES: CC(C)n1ncc(-c2ccnc(N[C@H]3CC[C@H](N)CC3)n2)c1CC1CC1]

(1r,4r)-N¹-(4-(5-(cyclopropylmethyl)-1-isopropyl-1H-pyrazol-4-yl)pyrimidin-2-yl)cyclohexane-1,4-diamine A9;

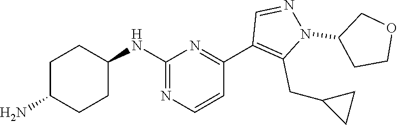

(1r,4S)-N¹-(4-(5-(cyclopropylmethyl)-1-((S)-tetrahydrofuran-3-yl)-1H-pyrazol-4-yl)pyrimidin-2-yl)cyclohexane-1,4-diamine A10;

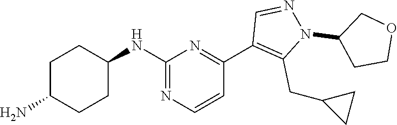

(1r,4S)-N¹-(4-(5-(cyclopropylmethyl)-1-((S)-tetrahydrofuran-3-yl)-1H-pyrazol-4-yl)pyrimidin-2-yl)cyclohexane-1,4-diamine A11;

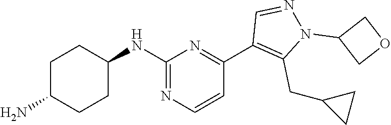 [SMILES: N[C@H]1CC[C@H](Nc2nccc(-c3cnn(C4COC4)c3CC3CC3)n2)CC1]

(1r,4r)-N¹-(4-(5-(cyclopropylmethyl)-1-(oxetan-3-yl)-1H-pyrazol-4-yl)pyrimidin-2-yl)cyclohexane-1,4-diamine A12;

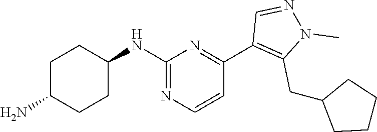 [SMILES: Cn1ncc(-c2ccnc(N[C@H]3CC[C@H](N)CC3)n2)c1CC1CCCC1]

(1r,4r)-N¹-(4-(5-(cyclopentylmethyl)-1-methyl-1H-pyrazol-4-yl)pyrimidin-2-yl)cyclohexane-1,4-diamine A13;

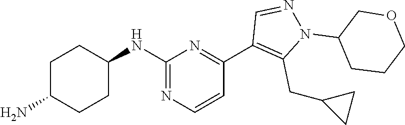 [SMILES: N[C@H]1CC[C@H](Nc2nccc(-c3cnn(C4CCCOC4)c3CC3CC3)n2)CC1]

(1r,4r)-N¹-(4-(5-(cyclopropylmethyl)-1-(tetrahydro-2H-pyran-3-yl)-1H-pyrazol-4-yl)pyrimidin-2-yl)cyclohexane-1,4-diamine A14;

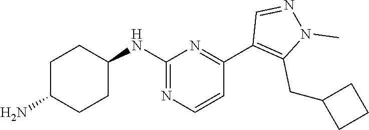 [SMILES: Cn1ncc(-c2ccnc(N[C@H]3CC[C@H](N)CC3)n2)c1CC1CCC1]

(1r,4r)-N¹-4-(5-(cyclobutylmethyl)-1-methyl-1H-pyrazol-4-yl)pyrimidin-2-yl)cyclohexane-1,4-diamine A15;

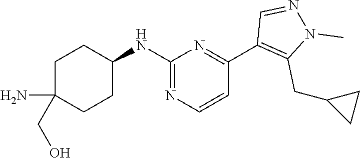

(1-amino-4-((4-(5-(cyclopropylmethyl)-1-methyl-1H-pyrazol-4-yl)pyrimidin-2-yl)amino)cyclohexyl)methanol A16;

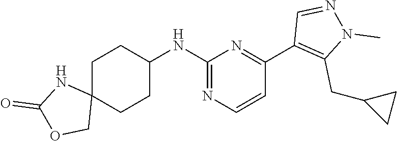 [SMILES: Cn1ncc(-c2ccnc(NC3CCC4(CC3)COC(=O)N4)n2)c1CC1CC1]

8-((4-(5-(cyclopropylmethyl)-1-methyl-1H-pyrazol-4-yl)pyrimidin-2-yl)amino)-3-oxa-1-azaspiro[4.5]decan-2-one A17;

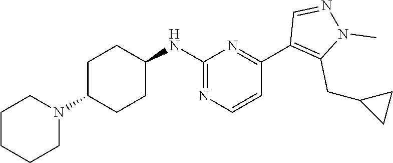 [SMILES: Cn1ncc(-c2ccnc(N[C@H]3CC[C@H](N4CCCCC4)CC3)n2)c1CC1CC1]

4-(5-(cyclopropylmethyl)-1-methyl-1H-pyrazol-4-yl)-N-((1r,4r)-4-(piperidin-1-yl)cyclohexyl)pyrimidin-2-amine A18;

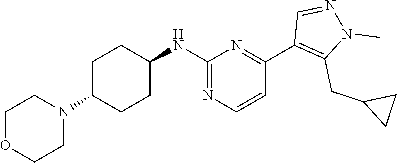 [SMILES: Cn1ncc(-c2ccnc(N[C@H]3CC[C@H](N4CCOCC4)CC3)n2)c1CC1CC1]

4-(5-(cyclopropylmethyl)-1-methyl-1H-pyrazol-4-yl)-N-((1r,4r)-4-morpholinocyclohexyl)-pyrimidin-2-amine A19;

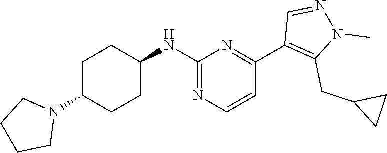 [SMILES: Cn1ncc(-c2ccnc(N[C@H]3CC[C@H](N4CCCC4)CC3)n2)c1CC1CC1]

4-(5-(cyclopropylmethyl)-1-methyl-1H-pyrazol-4-yl)-N-((1r,4r)-4-(pyrrolidin-1-yl)cyclohexyl)pyrimidin-2-amine A20;

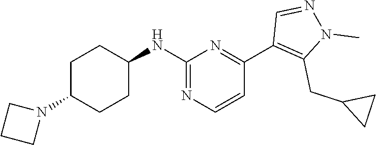 [SMILES: Cn1ncc(-c2ccnc(N[C@H]3CC[C@H](N4CCC4)CC3)n2)c1CC1CC1]

N-((1r,4r)-4-(azetidin-1-yl)cyclohexyl)-4-(5-(cyclopropylmethyl)-1-methyl-1H-pyrazol-4-yl)pyrimidin-2-amine A21;

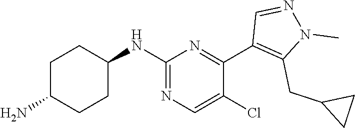 [SMILES: Cn1ncc(-c2nc(N[C@H]3CC[C@H](N)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)cyclohexane-1,4-diamine A22;

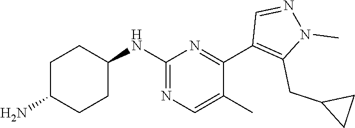 [SMILES: Cc1cnc(N[C@H]2CC[C@H](N)CC2)nc1-c1cnn(C)c1CC1CC1]

(1r,4r)-N¹-(4-(5-(cyclopropylmethyl)-1-methyl-1H-pyrazol-4-yl)-5-methylpyrimidin-2-yl)cyclohexane-1,4-diamine A23;

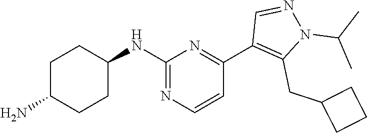 [SMILES: CC(C)n1ncc(-c2ccnc(N[C@H]3CC[C@H](N)CC3)n2)c1CC1CCC1]

(1r,4r)-N¹-(4-(5-(cyclobutylmethyl)-1-isopropyl-1H-pyrazol-4-yl)pyrimidin-2-yl)cyclohexane-1,4-diamine A24;

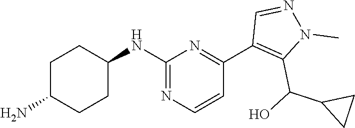 [SMILES: Cn1ncc(-c2ccnc(N[C@H]3CC[C@H](N)CC3)n2)c1C(O)C1CC1]

(4-(2-(((1r,4r)-4-aminocyclohexyl)amino)pyrimidin-4-yl)-1-methyl-1H-pyrazol-5-yl)(cyclopropyl)methanol A25;

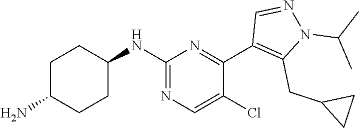 [SMILES: CC(C)n1ncc(-c2nc(N[C@H]3CC[C@H](N)CC3)ncc2Cl)c1CC1CC1]

(1r,4r)-N¹-(5-chloro-4-(5-(cyclopropylmethyl)-1-isopropyl-1H-pyrazol-4-yl)pyrimidin-2-yl)cyclohexane-1,4-diamine A26;

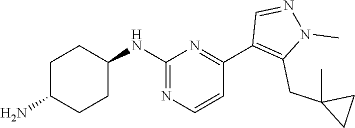 [SMILES: Cn1ncc(-c2ccnc(N[C@H]3CC[C@H](N)CC3)n2)c1CC1(C)CC1]

(1r,4r)-N¹-4-(1-methyl-5-((1-methylcyclopropyl)methyl)-1H-pyrazol-4-yl)pyrimidin-2-yl)cyclohexane-1,4-diamine A27;

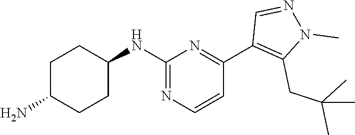 [SMILES: Cn1ncc(-c2ccnc(N[C@H]3CC[C@H](N)CC3)n2)c1CC(C)(C)C]

(1r,4r)-N¹-4-(1-methyl-5-neopentyl-1H-pyrazol-4-yl)pyrimidin-2-yl)cyclohexane-1,4-diamine A28;

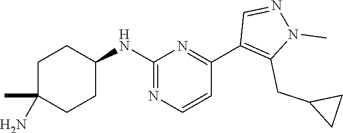

(1r,4r)-N¹-(4-(5-(cyclopropylmethyl)-1-methyl-1H-pyrazol-4-yl)pyrimidin-2-yl)-4-methylcyclohexane-1,4-diamine A29;

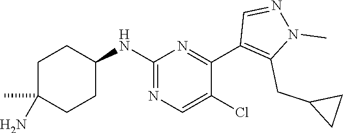

(1s,4s)-N¹-(5-chloro-4-(5-(cyclopropylmethyl)-1-methyl-1H-pyrazol-4-yl)pyrimidin-2-yl)-4-methylcyclohexane-1,4-diamine A30;

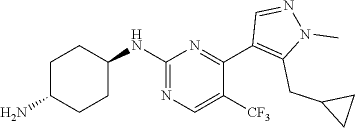

(1r,4r)-N¹-(4-(5-(cyclopropylmethyl)-1-methyl-1H-pyrazol-4-yl)-5-(trifluoromethyl)-pyrimidin-2-yl)cyclohexane-1,4-diamine A31;

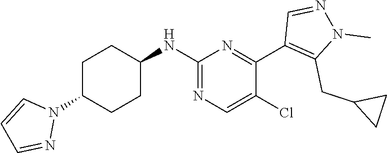 [SMILES: Cn1ncc(-c2nc(N[C@H]3CC[C@H](n4cccn4)CC3)ncc2Cl)c1CC1CC1]

N-((1r,4r)-4-(1H-pyrazol-1-yl)cyclohexyl)-5-chloro-4-(5-(cyclopropylmethyl)-1-methyl-1H-pyrazol-4-yl)pyrimidin-2-amine A32;

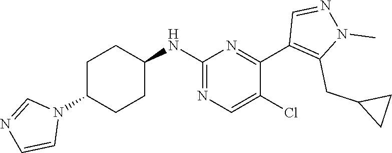 [SMILES: Cn1ncc(-c2nc(N[C@H]3CC[C@H](n4ccnc4)CC3)ncc2Cl)c1CC1CC1]

N-((1r,4r)-4-(1H-imidazol-1-yl)cyclohexyl)-5-chloro-4-(5-(cyclopropylmethyl)-1-methyl-1H-pyrazol-4-yl)pyrimidin-2-amine A33;

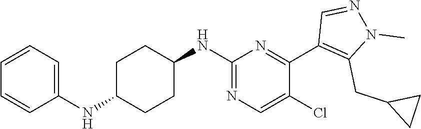 [SMILES: Cn1ncc(-c2nc(N[C@H]3CC[C@H](Nc4ccccc4)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A34;

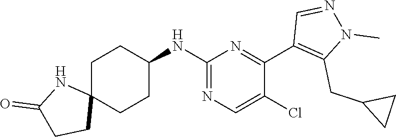

(5r,8r)-8-((5-chloro-4-(5-(cyclopropylmethyl)-1-methyl-1H-pyrazol-4-yl)pyrimidin-2-yl)amino)-1-azaspiro[4.5]decan-2-one A35;

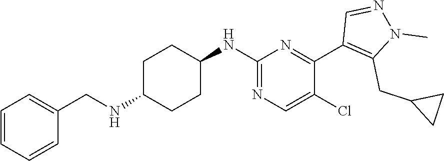 [SMILES: Cn1ncc(-c2nc(N[C@H]3CC[C@H](NCc4ccccc4)CC3)ncc2Cl)c1CC1CC1]

(1r,4r)-N¹-benzyl-N⁴-(5-chloro-4-(5-(cyclopropylmethyl)-1-methyl-1H-pyrazol-4-yl)pyrimidin-2-yl)cyclohexane-1,4-diamine A36;

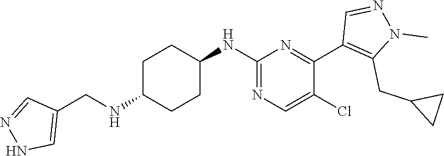 [SMILES: Cn1ncc(-c2nc(N[C@H]3CC[C@H](NCc4cn[nH]c4)CC3)ncc2Cl)c1CC1CC1]

(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A37;

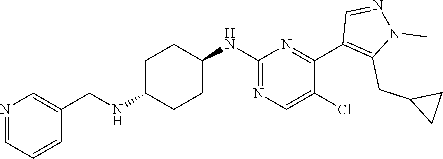 [SMILES: Cn1ncc(-c2nc(N[C@H]3CC[C@H](NCc4cccnc4)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-(pyridin-3-ylmethyl)cyclohexane-1,4-diamine A38;

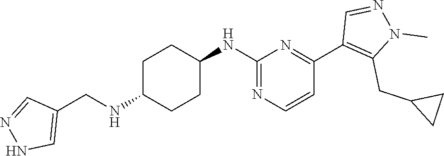 [SMILES: Cn1ncc(-c2ccnc(N[C@H]3CC[C@H](NCc4cn[nH]c4)CC3)n2)c1CC1CC1]

(1r,4r)-N¹-((1H-pyrazol-4-yl)methyl)-N⁴-(4-(5-(cyclopropylmethyl)-1-methyl-1H-pyrazol-4-yl)pyrimidin-2-yl)cyclohexane-1,4-diamine A39;

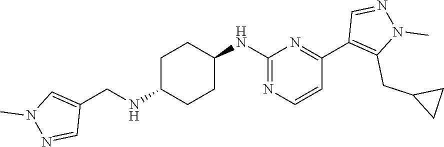 [SMILES: Cn1cc(CN[C@H]2CC[C@H](Nc3nccc(-c4cnn(C)c4CC4CC4)n3)CC2)cn1]

(1r,4r)-N¹-(4-(5-(cyclopropylmethyl)-1-methyl-1H-pyrazol-4-yl)pyrimidin-2-yl)-N⁴-((1-methyl-1H-pyrazol-4-yl)methyl)cyclohexane-1,4-diamine A40;

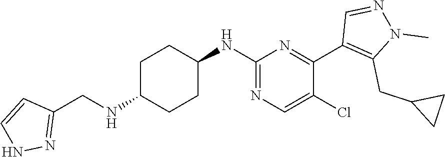 [SMILES: Cn1ncc(-c2nc(N[C@H]3CC[C@H](NCc4cc[nH]n4)CC3)ncc2Cl)c1CC1CC1]

(1r,4r)-N¹-((1H-pyrazol-5-yl)methyl)-N⁴-(5-chloro-4-(5-(cyclopropylmethyl)-1-methyl-1H-pyrazol-4-yl)pyrimidin-2-yl)cyclohexane-1,4-diamine A41;

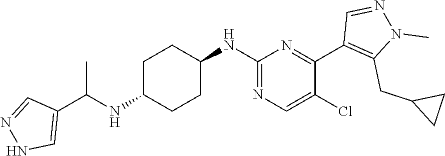 [SMILES: CC(N[C@H]1CC[C@H](Nc2ncc(Cl)c(-c3cnn(C)c3CC3CC3)n2)CC1)c1cn[nH]c1]

(1r,4r)-N¹-(1-(1H-pyrazol-4-yl)ethyl)-N⁴-(5-chloro-4-(5-(cyclopropylmethyl)-1-methyl-1H-pyrazol-4-yl)pyrimidin-2-yl)cyclohexane-1,4-diamine A42;

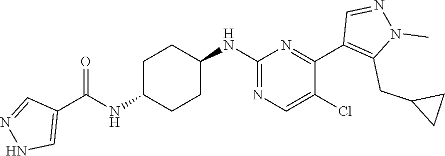 [SMILES: Cn1ncc(-c2nc(N[C@H]3CC[C@H](NC(=O)c4cn[nH]c4)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A43;

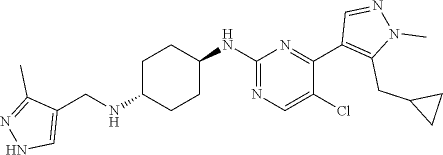 [SMILES: Cc1n[nH]cc1CN[C@H]1CC[C@H](Nc2ncc(Cl)c(-c3cnn(C)c3CC3CC3)n2)CC1]

(1r,4r)-N¹-(5-chloro-4-(5-(cyclopropylmethyl)-1-methyl-1H-pyrazol-4-yl)pyrimidin-2-yl)-N⁴-((5-methyl-1H-pyrazol-4-yl)methyl)cyclohexane-1,4-diamine A44;

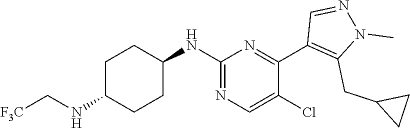 [SMILES: Cn1ncc(-c2nc(N[C@H]3CC[C@H](NCC(F)(F)F)CC3)ncc2Cl)c1CC1CC1]

(1r,4r)-N¹-(5-chloro-4-(5-(cyclopropylmethyl)-1-methyl-1H-pyrazol-4-yl)pyrimidin-2-yl)-N⁴-(2,2,2-trifluoroethyl)cyclohexane-1,4-diamine A45;

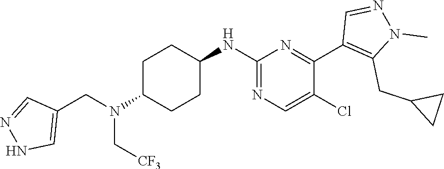 [SMILES: Cn1ncc(-c2nc(N[C@H]3CC[C@H](N(Cc4cn[nH]c4)CC(F)(F)F)CC3)ncc2Cl)c1CC1CC1]

(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)-N¹-(2,2,2-trifluoroethyl)cyclohexane-1,4-diamine A46;

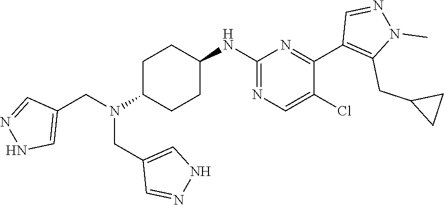 [SMILES: Cn1ncc(-c2nc(N[C@H]3CC[C@H](N(Cc4cn[nH]c4)Cc4cn[nH]c4)CC3)ncc2Cl)c1CC1CC1]

(1r,4r)-N¹,N¹-bis((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A47;

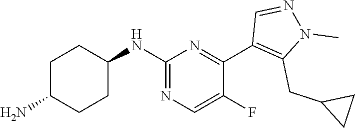 [SMILES: Cn1ncc(-c2nc(N[C@H]3CC[C@H](N)CC3)ncc2F)c1CC1CC1]

(1r,4r)-N¹-(4-(5-(cyclopropylmethyl)-1-methyl-1H-pyrazol-4-yl)-5-fluoropyrimidin-2-yl)cyclohexane-1,4-diamine A48;

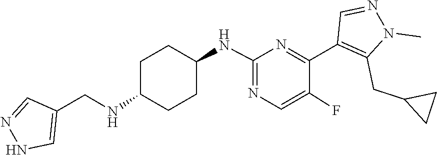 [SMILES: Cn1ncc(-c2nc(N[C@H]3CC[C@H](NCc4cn[nH]c4)CC3)ncc2F)c1CC1CC1]

(1r,4r)-N¹-((1H-pyrazol-4-yl)methyl)-N⁴-(4-(5-(cyclopropylmethyl)-1-methyl-1H-pyrazol-4-yl)-5-fluoropyrimidin-2-yl)cyclohexane-1,4-diamine A49;

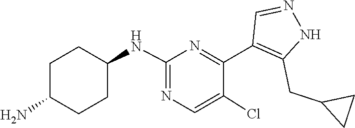 [SMILES: N[C@H]1CC[C@H](Nc2ncc(Cl)c(-c3cn[nH]c3CC3CC3)n2)CC1]

(1r,4r)-N¹-(5-chloro-4-(5-(cyclopropylmethyl)-1H-pyrazol-4-yl)pyrimidin-2-yl)cyclohexane-1,4-diamine A50;

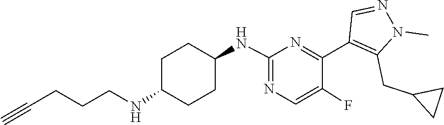 [SMILES: C#CCCCN[C@H]1CC[C@H](Nc2ncc(F)c(-c3cnn(C)c3CC3CC3)n2)CC1]

(1r,4r)-N¹-(4-(5-(cyclopropylmethyl)-1-methyl-1H-pyrazol-4-yl)-5-fluoropyrimidin-2-yl)-N⁴-(pent-4-yn-1-yl)cyclohexane-1,4-diamine A51;

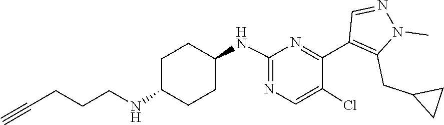 [SMILES: C#CCCCN[C@H]1CC[C@H](Nc2ncc(Cl)c(-c3cnn(C)c3CC3CC3)n2)CC1]

(1r,4r)-N¹-(4-(5-(cyclopropylmethyl)-1-methyl-1H-pyrazol-4-yl)-5-chloropyrimidin-2-yl)-N⁴-(pent-4-yn-1-yl)cyclohexane-1,4-diamine A52;

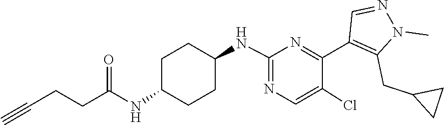 [SMILES: C#CCCC(=O)N[C@H]1CC[C@H](Nc2ncc(Cl)c(-c3cnn(C)c3CC3CC3)n2)CC1]

N-((1r,4r)-4-((4-(5-(cyclopropylmethyl)-1-methyl-1H-pyrazol-4-yl)-5-chloropyrimidin-2-yl)amino)cyclohexyl)pent-4-ynamide A53;

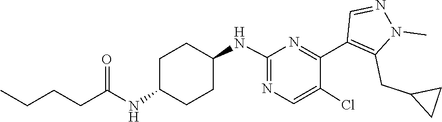 [SMILES: CCCCC(=O)N[C@H]1CC[C@H](Nc2ncc(Cl)c(-c3cnn(C)c3CC3CC3)n2)CC1]

N-((1r,4r)-4-((5-chloro-4-(5-(cyclopropylmethyl)-1-methyl-1H-pyrazol-4-yl)pyrimidin-2-yl)amino)cyclohexyl)pentanamide A54;

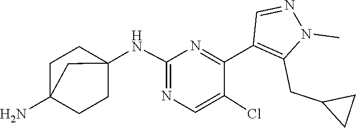 [SMILES: Cn1ncc(-c2nc(NC34CCC(N)(CC3)C4)ncc2Cl)c1CC1CC1]

N¹-(5-chloro-4-(5-(cyclopropylmethyl)-1-methyl-1H-pyrazol-4-yl)pyrimidin-2-yl)bicyclo[2.2.1]heptane-1,4-diamine A55;

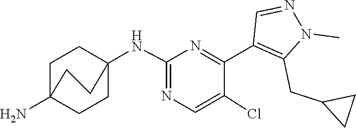 [SMILES: Cn1ncc(-c2nc(NC34CCC(N)(CC3)CC4)ncc2Cl)c1CC1CC1]

N¹-(5-chloro-4-(5-(cyclopropylmethyl)-1-methyl-1H-pyrazol-4-yl)pyrimidin-2-yl)bicyclo[2.2.2]octane-1,4-diamine A56;

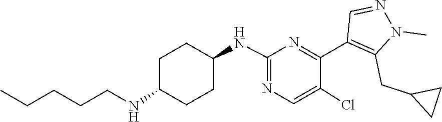 [SMILES: CCCCCN[C@H]1CC[C@H](Nc2ncc(Cl)c(-c3cnn(C)c3CC3CC3)n2)CC1]

(1r,4r)-N¹-(5-chloro-4-(5-(cyclopropylmethyl)-1-methyl-1H-pyrazol-4-yl)pyrimidin-2-yl)-N⁴-pentylcyclohexane-1,4-diamine A57;

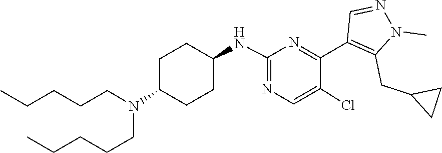 [SMILES: CCCCCN(CCCCC)[C@H]1CC[C@H](Nc2ncc(Cl)c(-c3cnn(C)c3CC3CC3)n2)CC1]

(1r,4r)-N¹-(5-chloro-4-(5-(cyclopropylmethyl)-1-methyl-1H-pyrazol-4-yl)pyrimidin-2-yl)-N⁴,N⁴-dipentylcyclohexane-1,4-diamine A58;

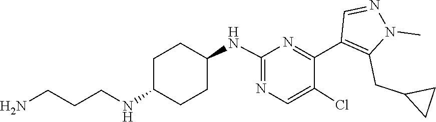 [SMILES: Cn1ncc(-c2nc(N[C@H]3CC[C@H](NCCCN)CC3)ncc2Cl)c1CC1CC1]

(1r,4r)-N¹-(3-aminopropyl)-N⁴-(5-chloro-4-(5-(cyclopropylmethyl)-1-methyl-1H-pyrazol-4-yl)pyrimidin-2-yl)cyclohexane-1,4-diamine A59;

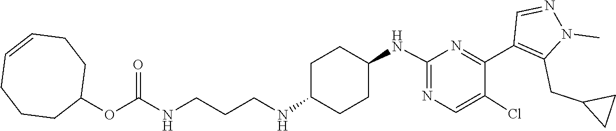 [SMILES: Cn1ncc(-c2nc(N[C@H]3CC[C@H](NCCCNC(=O)OC4CC/C=C\CCC4)CC3)ncc2Cl)c1CC1CC1]

(Z)-cyclooct-4-en-1-yl (3-(((1r,4r)-4-((5-chloro-4-(5-(cyclopropylmethyl)-1-methyl-1H-pyrazol-4-yl)pyrimidin-2-yl)amino)cyclohexyl)amino)propyl)carbamate A60;

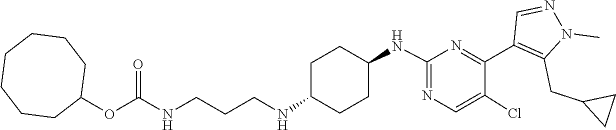 [SMILES: Cn1ncc(-c2nc(N[C@H]3CC[C@H](NCCCNC(=O)OC4CCCCCCC4)CC3)ncc2Cl)c1CC1CC1]

cyclooctyl (3-(((1r,4r)-4-((5-chloro-4-(5-(cyclopropylmethyl)-1-methyl-1H-pyrazol-4-yl)pyrimidin-2-yl)amino)cyclohexyl)amino)propyl)carbamate A61; or

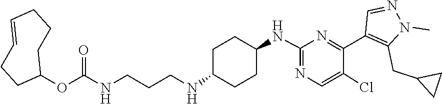 [SMILES: Cn1ncc(-c2nc(N[C@H]3CC[C@H](NCCCNC(=O)OC4CC/C=C/CCC4)CC3)ncc2Cl)c1CC1CC1]

(E)-cyclooct-4-en-1-yl (3-(((1r,4r)-4-((5-chloro-4-(5-(cyclopropylmethyl)-1-methyl-1H-pyrazol-4-yl)pyrimidin-2-yl)amino)cyclohexyl)amino)propyl)carbamate A62;
or a tautomer, a mixture of two or more tautomers, or an isotopic variant thereof; or a pharmaceutically acceptable salt, solvate, hydrate, or prodrug thereof.

In yet another embodiment, described herein is (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically acceptable salt, solvate, hydrate, or prodrug thereof.

In yet another embodiment, described herein is a p-toluensulfonate of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically solvate or hydrate.

In yet another embodiment, described herein is a di-p-toluensulfonate of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically solvate or hydrate.

In certain embodiments, the di-p-toluenesulfonate salt described herein is crystalline. In certain embodiments, the crystalline di-p-toluenesulfonate salt described herein has an X-ray powder diffractogram comprising peaks at two-theta angles (°) of approximately 5.5, 7.6, and 21.9. In certain embodiments, the crystalline di-p-toluenesulfonate salt described herein has an X-ray powder diffractogram comprising peaks at two-theta angles (°) of approximately 5.5, 7.6, 12.9, 17.3, 21.9, 22.3, 22.5, and 23.8. In certain embodiments, the crystalline di-p-toluenesulfonate salt described herein has an X-ray powder diffractogram comprising peaks at two-theta angles (°) of approximately 5.5, 7.6, 12.9, 14.9, 16.2, 17.3, 18.4, 18.6, 21.5, 21.9, 22.3, 22.5, 23.4, 23.8, 24.1, 26.2, 26.9, 27.0, and 28.8. In certain embodiments, the crystalline di-p-toluenesulfonate salt described herein has an X-ray powder diffractogram comprising peaks at two-theta angles (°) of approximately 5.5, 6.1, 7.6, 12.9, 14.9, 16.2, 17.3, 18.4, 18.6, 21.5, 21.9, 22.3, 22.5, 23.4, 23.8, 24.1, 26.2, 26.9, 27.0, and 28.8.

In certain embodiments, the crystalline di-p-toluenesulfonate salt described herein has a DSC thermogram comprising an endothermic peak at about 228° C. In certain embodiments, the crystalline di-p-toluenesulfonate salt described herein has a DSC thermogram comprising an endothermic peak at 228±3° C. In certain embodiments, the crystalline di-p-toluenesulfonate salt described herein is not hygroscopic. In certain embodiments, the crystalline di-p-toluenesulfonate salt described herein is unsolvated. In certain embodiments, the crystalline di-p-toluenesulfonate salt described herein has a solubility of about 2 mg/mL in water at 25° C. In certain embodiments, the crystalline di-p-toluenesulfonate salt described herein is nonhygroscopic.

Additional pharmaceutically acceptable salts of compound A22 and crystalline forms thereof are described in WO 2020/247345 A1, the disclosure of which is incorporated herein by reference in its entirety.

In still another embodiment, described herein is (1r,4r)-N¹-(5-chloro-4-(5-(cyclopropylmethyl)-1H-pyrazol-4-yl)pyrimidin-2-yl)cyclohexane-1,4-diamine A50, or a tautomer, a mixture of two or more tautomers, or an isotopic variant thereof; or a pharmaceutically acceptable salt, solvate, hydrate, or prodrug thereof.

In certain embodiments, a compound described herein is deuterium-enriched. In certain embodiments, a compound described herein is carbon-13 enriched. In certain embodiments, a compound described herein is carbon-14 enriched. In certain embodiments, a compound described herein contains one or more less prevalent isotopes for other elements, including, but not limited to, ¹⁵N for nitrogen; ¹⁷O or ¹⁸O for oxygen, and ³³S, ³⁴S, or ³⁶S for sulfur.

In certain embodiments, a compound described herein has an isotopic enrichment factor of no less than about 5, no less than about 10, no less than about 20, no less than about 30, no less than about 40, no less than about 50, no less than about 60, no less than about 70, no less than about 80, no less than about 90, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6410 for deuterium and 90 for carbon-13.

In certain embodiments, a compound described herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6,280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy.

In certain embodiments, a compound described herein has a carbon-13 enrichment factor of no less than about 1.8 (about 2% carbon-13 enrichment), no less than about 4.5 (about 5% carbon-13 enrichment), no less than about 9 (about 10% carbon-13 enrichment), no less than about 18 (about 20% carbon-13 enrichment), no less than about 45 (about 50% carbon-13 enrichment), no less than about 68 (about 75% carbon-13 enrichment), no less than about 72 (about 80% carbon-13 enrichment), no less than about 77 (about 85% carbon-13 enrichment), no less than about 81 (about 90% carbon-13 enrichment), no less than about 86 (about 95% carbon-13 enrichment), no less than about 87 (about 97% carbon-13 enrichment), no less than about 88 (about 98% carbon-13 enrichment), no less than about 89 (about 99% carbon-13 enrichment), or no less than about 90 (about 99.5% carbon-13 enrichment). The carbon-13 enrichment can be determined using conventional analytical methods known to one of ordinary skill in the art, including mass spectrometry and nuclear magnetic resonance spectroscopy.

In certain embodiments, at least one of the atoms of a compound described herein, as specified as isotopically enriched, has isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as isotopically enriched, have isotopic enrichment of no less than about 1%, no less than about 2%, no less than about 5%, no less than about 10%, no less than about 20%, no less than about 50%, no less than about 70%, no less than about 80%, no less than about 90%, or no less than about 98%. In any events, the isotopic enrichment of the isotopically enriched atom of a compound described herein is no less than the natural abundance of the isotope specified.

In certain embodiments, at least one of the atoms of a compound described herein, as specified as deuterium-enriched, has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as deuterium-enriched, have deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, at least one of the atoms of a compound described herein, as specified as ¹³C-enriched, has carbon-13 enrichment of no less than about 2%, no less than about 5%, no less than about 10%, no less than about 20%, no less than about 50%, no less than about 70%, no less than about 80%, no less than about 90%, or no less than about 98%. In certain embodiments, the atoms of a compound described herein, as specified as ¹³C-enriched, have carbon-13 enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, a compound described herein is isolated or purified. In certain embodiments, a compound described herein has a purity of at least about 50%, at least about 70%, at least about 80%, at least about 90%, at least about 95%, at least about 98%, at least about 99%, or at least about 99.5% by weight.

The compounds described herein are intended to encompass all possible stereoisomers unless a particular stereochemistry is specified. Where a compound described herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

A compound described herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound described herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; Wiley-VCH and VHCA, Zurich, 2011. In certain embodiments, a pharmaceutically acceptable salt of a compound described herein is a hydrate.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid. In certain embodiments, a compound described herein is a hydrochloride salt. In certain embodiments, a compound described herein is a p-toluenesulfonate salt. In certain embodiments, a compound described herein is a di-p-toluenesulfonate salt.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound described herein may also be provided as a prodrug, which is a functional derivative of a compound, for example, of Formula I and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

The compounds described herein can be prepared, isolated, or obtained by any method known to one of ordinary skill in the art, for example, by following the procedures described in U.S. Pat. No. 10,376,511 B2 and WO 2019/155468 A1, the disclosure of each of which is incorporated herein by reference in its entirety.

### Pharmaceutical Compositions

In one embodiment, provided herein is a pharmaceutical composition, comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

A pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, FL, 2008.

In one embodiment, a pharmaceutical composition provided herein is formulated in a dosage form for oral administration. In another embodiment, a pharmaceutical composition provided herein is formulated in a dosage form for parenteral administration. In yet another embodiment, a pharmaceutical composition provided herein is formulated in a dosage form for intravenous administration. In yet another embodiment, a pharmaceutical composition provided herein is formulated in a dosage form for intramuscular administration. In yet another embodiment, a pharmaceutical composition provided herein is formulated in a dosage form for subcutaneous administration. In still another embodiment, a pharmaceutical composition provided herein is formulated in a dosage form for topical administration.

A pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

A pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

In one embodiment, a pharmaceutical composition provided herein comprises a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and sugar beads, talc, and povidone.

In another embodiment, a pharmaceutical composition provided herein comprises compound A22, or a tautomer, a mixture of two or more tautomers, or an isotopic variant thereof; or a pharmaceutically acceptable salt, solvate, hydrate, or prodrug thereof; and sugar beads, talc, and povidone.

In yet another embodiment, a pharmaceutical composition provided herein comprises compound A22 or a pharmaceutically acceptable salt; and sugar beads, talc, and povidone. In one embodiment, the pharmaceutical composition is formulated as a capsule.

In yet another embodiment, a pharmaceutical composition provided herein comprises a pharmaceutically acceptable salt of compound A22; and sugar beads, talc, and povidone. In one embodiment, the pharmaceutical composition is formulated as a capsule.

In certain embodiments, a pharmaceutical composition provided herein comprises compound A22 or a pharmaceutically acceptable salt in an amount ranging from about 0.1 to about 50, from about 0.2 to about 20, from about 0.5 to about 10, or from about 0.5 to about 5 mg per capsule. In certain embodiments, a pharmaceutical composition provided herein comprises compound A22 or a pharmaceutically acceptable salt in an amount ranging from about 0.1 to about 50 mg per capsule. In certain embodiments, a pharmaceutical composition provided herein comprises compound A22 or a pharmaceutically acceptable salt in an amount ranging from about 0.2 to about 20 mg per capsule. In certain embodiments, a pharmaceutical composition provided herein comprises compound A22 or a pharmaceutically acceptable salt in an amount ranging from about 0.5 to about 10 mg per capsule. In certain embodiments, a pharmaceutical composition provided herein comprises compound A22 or a pharmaceutically acceptable salt in an amount ranging from about 0.5 to about 5 mg per capsule.

In certain embodiments, a pharmaceutical composition provided herein comprises compound A22 or a pharmaceutically acceptable salt in an amount of about 0.5, about 0.6, about 0.7, about 0.8, about 0.9, about 1, about 1.2, about 1.4, about 1.6, about 1.8, about 2, about 2.5, about 3, about 3.5, about 4, about 4.5, about 5, about 6, about 7, about 8, about 9, about 10, about 12, about 15, about 17, or about 20 mg per capsule. In certain embodiments, a pharmaceutical composition provided herein comprises compound A22 or a pharmaceutically acceptable salt in an amount of about 0.5, about 1, about 2, or about 7 mg per capsule.

In yet another embodiment, a pharmaceutical composition provided herein comprises a p-toluenesulfonate salt of compound A22; and sugar beads, talc, and povidone. In one embodiment, the pharmaceutical composition is formulated as a capsule.

In certain embodiments, a pharmaceutical composition provided herein comprises a p-toluenesulfonate salt of compound A22 in an amount ranging from about 0.1 to about 50, from about 0.2 to about 20, from about 0.5 to about 10, or from about 0.5 to about 5 mg per capsule. In certain embodiments, a pharmaceutical composition provided herein comprises a p-toluenesulfonate salt of compound A22 in an amount ranging from about 0.1 to about 50 mg per capsule. In certain embodiments, a pharmaceutical composition provided herein comprises a p-toluenesulfonate salt of compound A22 in an amount ranging from about 0.2 to about 20 mg per capsule. In certain embodiments, a pharmaceutical composition provided herein comprises a p-toluenesulfonate salt of compound A22 in an amount ranging from about 0.5 to about 10 mg per capsule. In certain embodiments, a pharmaceutical composition provided herein comprises a p-toluenesulfonate salt of compound A22 in an amount ranging from about 0.5 to about 5 mg per capsule.

In certain embodiments, a pharmaceutical composition provided herein comprises a p-toluenesulfonate salt of compound A22 in an amount of about 0.5, about 0.6, about 0.7, about 0.8, about 0.9, about 1, about 1.2, about 1.4, about 1.6, about 1.8, about 2, about 2.5, about 3, about 3.5, about 4, about 4.5, about 5, about 6, about 7, about 8, about 9, about 10, about 12, about 15, about 17, or about 20 mg per capsule. In certain embodiments, a pharmaceutical composition provided herein comprises a p-toluenesulfonate salt of compound A22 in an amount of about 0.5, about 1, about 2, or about 7 mg per capsule.

In still another embodiment, a pharmaceutical composition provided herein comprises a di-p-toluenesulfonate salt of compound A22; and sugar beads, talc, and povidone. In one embodiment, the pharmaceutical composition is formulated as a capsule.

In certain embodiments, a pharmaceutical composition provided herein comprises a di-p-toluenesulfonate salt of compound A22 in an amount ranging from about 0.1 to about 50, from about 0.2 to about 20, from about 0.5 to about 10, or from about 0.5 to about 5 mg per capsule. In certain embodiments, a pharmaceutical composition provided herein comprises a di-p-toluenesulfonate salt of compound A22 in an amount ranging from about 0.1 to about 50 mg per capsule. In certain embodiments, a pharmaceutical composition provided herein comprises a di-p-toluenesulfonate salt of compound A22 in an amount ranging from about 0.2 to about 20 mg per capsule. In certain embodiments, a pharmaceutical composition provided herein comprises a di-p-toluenesulfonate salt of compound A22 in an amount ranging from about 0.5 to about 10 mg per capsule. In certain embodiments, a pharmaceutical composition provided herein comprises a di-p-toluenesulfonate salt of compound A22 in an amount ranging from about 0.5 to about 5 mg per capsule.

In certain embodiments, a pharmaceutical composition provided herein comprises a di-p-toluenesulfonate salt of compound A22 in an amount of about 0.5, about 0.6, about 0.7, about 0.8, about 0.9, about 1, about 1.2, about 1.4, about 1.6, about 1.8, about 2, about 2.5, about 3, about 3.5, about 4, about 4.5, about 5, about 6, about 7, about 8, about 9, about 10, about 12, about 15, about 17, or about 20 mg per capsule. In certain embodiments, a pharmaceutical composition provided herein comprises a di-p-toluenesulfonate salt of compound A22 in an amount of about 0.5, about 1, about 2, or about 7 mg per capsule.

In certain embodiments, a pharmaceutical composition provided herein is formulated as an immediate-release capsule with a size of, e.g., size 1 or size 000.

### Methods of Treatment

In one embodiment, provided herein is method of treating a malignant solid tumor in a subject, comprising administering to the subject in need thereof a therapeutically effective amount of a compound described herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the malignant solid tumor is advanced. In certain embodiments, the malignant solid tumor is unresectable. In certain embodiments, the malignant solid tumor is inoperable. In certain embodiments, the malignant solid tumor is incurable. In certain embodiments, the malignant solid tumor is metastatic. In certain embodiments, the malignant solid tumor is recurrent. In certain embodiments, the malignant solid tumor is relapsed. In certain embodiments, the malignant solid tumor is refractory. In certain embodiments, the malignant solid tumor is refractory to a standard therapy. In certain embodiments, the malignant solid tumor is intolerant of a standard therapy. In certain embodiments, the malignant solid tumor is drug-resistant. In certain embodiments, the malignant solid tumor harbors an MYC aberration (e.g., MYC gene translocation, MYC gene amplification, MYC mRNA overexpression, and/or MYC protein overexpression). In certain embodiments, the malignant solid tumor harbors a c-MYC aberration (e.g., c-MYC gene translocation, c-MYC gene amplification, c-MYC mRNA overexpression, and/or c-MYC protein overexpression).

In certain embodiments, the malignant solid tumor is stage I. In certain embodiments, the malignant solid tumor is stage II. In certain embodiments, the malignant solid tumor is stage III. In certain embodiments, the malignant solid tumor is stage IV. In certain embodiments, the malignant solid tumor is stage II, III, or IV. In certain embodiments, the malignant solid tumor is stage III or IV.

In certain embodiments, the malignant solid tumor is bladder cancer, brain cancer, breast cancer, cervical cancer, colorectal cancer, endometrial cancer, esophageal cancer, gastric cancer, glioblastoma, head or neck cancer, hepatic cancer, lymphoma, lung cancer, melanoma, mesothelioma, non-Hodgkin's lymphoma, non-small cell lung cancer, nonmelanoma skin cancer, oral cancer, ovarian cancer, pancreatic cancer, prostate cancer, renal cancer, sarcoma, skin cancer, small cell lung cancer, thyroid cancer, or uterine cancer. In certain embodiments, the malignant solid tumor is bladder cancer, breast cancer, colorectal cancer, lung cancer, melanoma, non-Hodgkin's lymphoma, oral cancer, pancreatic cancer, prostate cancer, renal cancer, thyroid cancer, or uterine cancer.

In certain embodiments, the malignant solid tumor is bladder cancer. In certain embodiments, the malignant solid tumor is breast cancer. In certain embodiments, the malignant solid tumor is colorectal cancer. In certain embodiments, the malignant solid tumor is lung cancer. In certain embodiments, the malignant solid tumor is melanoma. In certain embodiments, the malignant solid tumor is oral cancer. In certain embodiments, the malignant solid tumor is pancreatic cancer. In certain embodiments, the malignant solid tumor is prostate cancer. In certain embodiments, the malignant solid tumor is renal cancer. In certain embodiments, the malignant solid tumor is thyroid cancer. In certain embodiments, the malignant solid tumor is uterine cancer.

In certain embodiments, the malignant solid tumor is lymphoma. In certain embodiments, the lymphoma is advanced. In certain embodiments, the lymphoma is unresectable. In certain embodiments, the lymphoma is inoperable. In certain embodiments, the lymphoma is incurable. In certain embodiments, the lymphoma is metastatic. In certain embodiments, the lymphoma is recurrent. In certain embodiments, the lymphoma is relapsed. In certain embodiments, the lymphoma is refractory. In certain embodiments, the lymphoma is refractory to a standard therapy. In certain embodiments, the lymphoma is intolerant of a standard therapy. In certain embodiments, the lymphoma is drug-resistant. In certain embodiments, the lymphoma harbors an MYC aberration (e.g., MYC gene translocation, MYC gene amplification, MYC mRNA overexpression, and/or MYC protein overexpression). In certain embodiments, the lymphoma harbors a c-MYC aberration (e.g., c-MYC gene translocation, c-MYC gene amplification, c-MYC mRNA overexpression, and/or c-MYC protein overexpression).

In certain embodiments, the lymphoma is stage I. In certain embodiments, the lymphoma is stage II. In certain embodiments, the lymphoma is stage III. In certain embodiments, the lymphoma is stage IV. In certain embodiments, the lymphoma is stage II, III, or IV. In certain embodiments, the lymphoma is stage III or IV.

In certain embodiments, the malignant solid tumor is non-Hodgkin's lymphoma. In certain embodiments, the non-Hodgkin's lymphoma is advanced. In certain embodiments, the non-Hodgkin's lymphoma is unresectable. In certain embodiments, the non-Hodgkin's lymphoma is inoperable. In certain embodiments, the non-Hodgkin's lymphoma is incurable. In certain embodiments, the non-Hodgkin's lymphoma is metastatic. In certain embodiments, the non-Hodgkin's lymphoma is recurrent. In certain embodiments, the non-Hodgkin's lymphoma is relapsed. In certain embodiments, the lymphoma is refractory. In certain embodiments, the non-Hodgkin's lymphoma is refractory to a standard therapy. In certain embodiments, the non-Hodgkin's lymphoma is intolerant of a standard therapy. In certain embodiments, the non-Hodgkin's lymphoma is drug-resistant. In certain embodiments, the lymphoma is drug-resistant. In certain embodiments, the non-Hodgkin's lymphoma harbors an MYC aberration (e.g., MYC gene translocation, MYC gene amplification, MYC mRNA overexpression, and/or MYC protein overexpression). In certain embodiments, the non-Hodgkin's lymphoma harbors a c-MYC aberration (e.g., c-MYC gene translocation, c-MYC gene amplification, c-MYC mRNA overexpression, and/or c-MYC protein overexpression).

In certain embodiments, the non-Hodgkin's lymphoma is stage I. In certain embodiments, the non-Hodgkin's lymphoma is stage II. In certain embodiments, the non-Hodgkin's lymphoma is stage III. In certain embodiments, the non-Hodgkin's lymphoma is stage IV. In certain embodiments, the non-Hodgkin's lymphoma is stage II, III, or IV. In certain embodiments, the non-Hodgkin's lymphoma is stage III or IV.

In certain embodiments, the non-Hodgkin's lymphoma is B-cell non-Hodgkin's lymphoma. In certain embodiments, the B-cell non-Hodgkin's lymphoma is diffuse large B-cell lymphoma (DLBCL), follicular lymphoma, mantle cell lymphoma, or Burkitt lymphoma. In certain embodiments, the non-Hodgkin's lymphoma is T-cell non-Hodgkin's lymphoma. In certain embodiments, the non-Hodgkin's lymphoma is NK-cell non-Hodgkin's lymphoma.

In certain embodiments, the subject has failed a prior therapy. In certain embodiments, the subject has failed more than one prior therapy.

In certain embodiments, the subject is a mammal. In certain embodiments, the subject is a human. In certain embodiments, the subject is an adult human. In certain embodiments, the subject is a pediatric human.

A method provided herein encompasses treating a subject regardless of patient's age, although some diseases are more common in certain age groups.

In certain embodiments, the therapeutically effective amount of a compound described herein, e.g., compound A22, is ranging from about 0.01 to about 10 mg/kg per day, from about 0.02 to about 5 mg/kg per day, from about 0.05 to about 2 mg/kg per day, or from about 0.1 to about 1 mg/kg per day. In one embodiment, the therapeutically effective amount of a compound described herein, e.g., compound A22, is ranging from about 0.01 to about 10 mg/kg per day. In another embodiment, the therapeutically effective amount of a compound described herein, e.g., compound A22, is ranging from about 0.02 to about 5 mg/kg per day. In yet another embodiment, the therapeutically effective amount of a compound described herein, e.g., compound A22, is ranging from about 0.05 to about 2 mg/kg per day. In yet another embodiment, the therapeutically effective amount of a compound described herein, e.g., compound A22, is ranging from about 0.1 to about 1 mg/kg per day. In still another embodiment, the therapeutically effective amount of a compound described herein, e.g., compound A22, is about 0.1, about 0.2, about 0.3, about 0.4, about 0.5, about 0.6, about 0.7, about 0.8, about 0.9, about 1, about 2, about 3, about 4, about 5, about 6, about 7, about 8, about 9, or about 10 mg/kg per day.

In certain embodiments, the therapeutically effective amount of a compound described herein, e.g., compound A22, is ranging from about 1 to about 500 mg per day, from about 2 to about 200 mg per day, from about 5 to about 100 mg per day, or from about 10 mg to about 100 mg per day. In one embodiment, the therapeutically effective amount of a compound described herein, e.g., compound A22, is ranging from about 1 to about 500 mg per day. In another embodiment, the therapeutically effective amount of a compound described herein, e.g., compound A22, is ranging from about 2 to about 200 mg per day. In yet another embodiment, the therapeutically effective amount of a compound described herein, e.g., compound A22, is ranging from about 5 to about 100 mg per day. In yet another embodiment, the therapeutically effective amount of a compound described herein, e.g., compound A22, is ranging from about 10 to about 100 mg per day. In still another embodiment, the therapeutically effective amount of a compound described herein, e.g., compound A22, is about 10, about 20, about 30, about 40, about 50, about 60, about 70, about 80, about 90, about 100, about 150, or about 200 mg per day.

It is understood that the administered dose of a compound described herein can also be expressed in units other than mg/kg every other day. For example, doses for parenteral administration can be expressed as mg/m² per day. One of ordinary skill in the art would readily know how to convert doses from mg/kg per day to mg/m² per day to given either the height or weight of a subject or both. For example, a dose of 1 mg/m² per day for a 65 kg human is approximately equal to 58 mg/kg per day.

Depending on the disease to be treated and the subject's condition, a compound described herein may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration.

In one embodiment, a compound described herein, e.g., compound A22, is administered orally. In another embodiment, a compound described herein, e.g., compound A22, is administered parenterally. In yet another embodiment, a compound described herein, e.g., compound A22, is administered intravenously. In yet another embodiment, a compound described herein, e.g., compound A22, is administered intramuscularly. In yet another embodiment, a compound described herein, e.g., compound A22, is administered subcutaneously. In still another embodiment, a compound described herein, e.g., compound A22, is administered topically.

A compound described herein, e.g., compound A22, can be delivered as a single dose such as, e.g., a single bolus injection, or oral tablets or pills; or over time such as, e.g., continuous infusion over time or divided bolus doses over time. A compound described herein, e.g., compound A22, can be administered repetitively if necessary, for example, until the subject experiences stable disease or regression, or until the subject experiences disease progression or unacceptable toxicity. Stable disease or lack thereof is determined by a method known in the art such as evaluation of subject's symptoms, physical examination, visualization of the cancer that has been imaged using X-ray, CAT, PET, or MRI scan and other commonly accepted evaluation modalities.

A compound described herein, e.g., compound A22, can be administered once daily (QD), or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound described herein, e.g., compound A22, is administration for one to six days per week, administration in cycles (e.g., daily administration for two to eight consecutive weeks, then a rest period with no administration for up to one week), or administration on alternate days.

It will be understood, however, that the specific dose level and frequency of dosage for any particular subject can be varied and will depend upon a variety of factors including the activity of the specific compound employed, e.g., compound A22, the metabolic stability and length of action of the compound, the age, body weight, general health, sex, diet, mode and time of administration, rate of excretion, drug combination, the severity of the particular condition, and the host undergoing therapy.

In certain embodiments, a compound described herein, e.g., compound A22, is cyclically administered to a subject to be treated. Cycling therapy involves the administration of the compound for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

Consequently, in one embodiment, a compound described herein, e.g., compound A22, is administered for a cycle of about one week, about two weeks, about three weeks, about four weeks, about five weeks, about six weeks, about eight weeks, or about ten weeks, with a rest period of about 1 day to about four weeks. In one embodiment, a compound described herein, e.g., compound A22, is administered for a cycle of three weeks, four weeks, five weeks, or six weeks with a rest period of 1, 3, 5, 7, 9, 12, or 14. In certain embodiments, the rest period is 7 days. In certain embodiments, the rest period is 14 days. In certain embodiments, the rest period is a period that is sufficient for bone marrow recovery. The frequency, number, and length of dosing cycles can be increased or decreased.

In one embodiment, a compound described herein, e.g., compound A22, is administered for three weeks in a 28-day cycle with a 7-day rest period. In one embodiment, in a 28-day cycle with a 7-day rest period, a compound described herein, e.g., compound A22, is administered every day for five days of a week. In another embodiment, in a 28-day cycle with a 7-day rest period, a compound described herein, e.g., compound A22, is administered on Days 1, 2, 3, 4, 5, 8, 9, 10, 11, 12, 15, 16, 17, 18, and 19. In one embodiment, in a 28-day cycle with a 7-day rest period, a compound described herein, e.g., compound A22, is administered every day for three days of a week. In another embodiment, in a 28-day cycle with a 7-day rest period, a compound described herein, e.g., compound A22, is administered on Days 1, 3, 5, 8, 10, 12, 15, 17, and 19.

In certain embodiments, the subject is treated with a compound described herein, e.g., compound A22, from about 1 to about 50, from about 2 to about 20, from about 2 to 10, or from about 4 to about 8 cycles. In certain embodiments, the subject is treated with a compound described herein, e.g., compound A22, from about 1 to about 50 cycles. In certain embodiments, the subject is treated with a compound described herein, e.g., compound A22, from about 2 to about 20 cycles. In certain embodiments, the subject is treated with a compound described herein, e.g., compound A22, from about 2 to 10 cycles. In certain embodiments, the subject is treated with a compound described herein, e.g., compound A22, from about 4 to about 8 cycles.

In one embodiment, provided herein is a method of inhibiting the growth of a cell, comprising contacting the cell with an effective amount of a compound described herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is a method of inducing apoptosis in a cell, comprising contacting the cell with an effective amount of a compound described herein, e.g., a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a malignant solid tumor cell. In certain embodiments, the cell is an advanced malignant solid tumor cell. In certain embodiments, the cell is an unresectable malignant solid tumor cell. In certain embodiments, the cell is an inoperable malignant solid tumor cell. In certain embodiments, the cell is an incurable malignant solid tumor cell. In certain embodiments, the cell is a metastatic malignant solid tumor cell. In certain embodiments, the cell is a recurrent malignant solid tumor cell. In certain embodiments, the cell is a relapsed malignant solid tumor cell. In certain embodiments, the cell is a refractory malignant solid tumor cell. In certain embodiments, the cell is a cell of a malignant solid tumor refractory to a standard therapy. In certain embodiments, the cell is a cell of a malignant solid tumor intolerant of a standard therapy. In certain embodiments, the cell is a drug-resistant malignant solid tumor cell. In certain embodiments, the cell is a malignant solid tumor cell harboring an MYC aberration (e.g., MYC gene translocation, MYC gene amplification, MYC mRNA overexpression, and/or MYC protein overexpression). In certain embodiments, the cell is a malignant solid tumor cell harboring a c-MYC aberration (e.g., c-MYC gene translocation, c-MYC gene amplification, c-MYC mRNA overexpression, and/or c-MYC protein overexpression).

In certain embodiments, the cell is a lymphoma cell. In certain embodiments, the cell is an advanced lymphoma cell. In certain embodiments, the cell is an unresectable lymphoma cell. In certain embodiments, the cell is an inoperable lymphoma cell. In certain embodiments, the cell is an incurable lymphoma cell. In certain embodiments, the cell is a metastatic lymphoma cell. In certain embodiments, the cell is a recurrent lymphoma cell. In certain embodiments, the cell is a relapsed lymphoma cell. In certain embodiments, the cell is a refractory lymphoma cell. In certain embodiments, the cell is a cell of lymphoma refractory to a standard therapy. In certain embodiments, the cell is a cell of lymphoma intolerant of a standard therapy. In certain embodiments, the cell is a drug-resistant lymphoma cell. In certain embodiments, the cell is a lymphoma cell harboring an MYC aberration (e.g., MYC gene translocation, MYC gene amplification, MYC mRNA overexpression, and/or MYC protein overexpression). In certain embodiments, the cell is a lymphoma cell harboring a c-MYC aberration (e.g., c-MYC gene translocation, c-MYC gene amplification, c-MYC mRNA overexpression, and/or c-MYC protein overexpression).

In certain embodiments, the cell is a non-Hodgkin's lymphoma cell. In certain embodiments, the cell is an advanced non-Hodgkin's lymphoma cell. In certain embodiments, the cell is an unresectable non-Hodgkin's lymphoma cell. In certain embodiments, the cell is an inoperable non-Hodgkin's lymphoma cell. In certain embodiments, the cell is an incurable non-Hodgkin's lymphoma cell. In certain embodiments, the cell is a metastatic non-Hodgkin's lymphoma cell. In certain embodiments, the cell is a recurrent non-Hodgkin's lymphoma cell. In certain embodiments, the cell is a relapsed non-Hodgkin's lymphoma cell. In certain embodiments, the cell is a refractory non-Hodgkin's lymphoma cell. In certain embodiments, the cell is a cell of non-Hodgkin's lymphoma refractory to a standard therapy. In certain embodiments, the cell is a cell of non-Hodgkin's lymphoma intolerant of a standard therapy. In certain embodiments, the cell is a drug-resistant non-Hodgkin's lymphoma cell. In certain embodiments, the cell is a non-Hodgkin's lymphoma cell harboring an MYC aberration (e.g., MYC gene translocation, MYC gene amplification, MYC mRNA overexpression, and/or MYC protein overexpression). In certain embodiments, the cell is a non-Hodgkin's lymphoma cell harboring a c-MYC aberration (e.g., c-MYC gene translocation, c-MYC gene amplification, c-MYC mRNA overexpression, and/or c-MYC protein overexpression).

The disclosure will be further understood by the following non-limiting examples.

### EXAMPLES

As used herein, the symbols and conventions used in these processes, schemes, and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: mg (milligrams); mL (milliliters); μL (microliters); h (hour or hours); and min (minutes).

### Example 1

### Toxicology Studies of Compound A22

The toxicology studies of compound A22 were performed in Sprague Dawley rats and Beagle dogs. In GLP, 28-day (once daily, 7 days per week) studies, compound A22 was administered as a di-HCl salt or di-p-toluenesulfonate salt by oral gavage. The results are summarized in Tables 1 and 2, where HNSTD is highest nonseverely toxic dose; NOAEL is no-observed-adverse-effect-level; and STDio is severely toxic dose in 10%.

TABLE 1
Steady State Toxicokinetic Parameters
of Compound A22 in Sprague Dawley Rats

| | | Male | | Female | |
|---|---|---|---|---|---|
| Classifi- | Dose | Cₘₐₓ | AUC₀₋ₜ | Cₘₐₓ | AUC₀₋ₜ |
| cation | (mg/kg/day) | (ng/mL) | (ng · h/mL) | (ng/mL) | (ng · h/mL) |
| NOAEL | 0.7 | 13.7 | 152 | 13.6 | 160 |
| STD₁₀ | 2.5 | 184 | 2520 | 156 | 2390 |

TABLE 2
Steady State Toxicokinetic Parameters
of Compound A22 in Beagle Dogs

| | | Male | | Female | |
|---|---|---|---|---|---|
| Classifi- | Dose | Cₘₐₓ | AUC₀₋ₜ | Cₘₐₓ | AUC₀₋ₜ |
| cation | (mg/kg/day) | (ng/mL) | (ng · h/mL) | (ng/mL) | (ng · h/mL) |
| NOAEL | 0.06 | 6.38 | 102 | 7.55 | 129 |
| HNSTD | 0.2 | 34.4 | 567 | 30.6 | 471 |

### Example 2

### An Open-Label, Escalating Multiple-Dose Study to Evaluate the Safety, Toxicity, and Pharmacokinetics of Compound A22 in Subjects with an Advanced Solid Tumor or Non-Hodgkin Lymphoma

This is a multicenter, open label, nonrandomized, sequential dose escalation/cohort expansion, multiple dose study, evaluating the safety, toxicity, and pharmacokinetics (PK) as well as efficacy of (1r,4r)-N¹-(5-chloro-4-(5-(cyclopropylmethyl)-1-methyl-1H-pyrazol-4-yl)pyrimidin-2-yl)cyclohexane-1,4-diamine) A22 as a di-p-toluenesulfonate salt in subjects with an advanced solid tumor or non-Hodgkin lymphoma (NHL). The study is conducted in two phases: Phases 1a and 1b.

Phase 1a is a dose escalation phase, determining the dose limiting toxicities (DLTs) and maximum tolerated dose (MTD) of orally administered compound A22 in subjects with an advanced solid tumor or NHL. The starting dose of compound A22 for Cohort 1 is ≤21 mg and does not exceed the highest dose deemed safe as determined in the BTX-A51-001 study (NCT04243785) of compound A22. The six dosing levels for Phase la (dose escalation) are listed in Table 3.

TABLE 3
Dosing Levels in Phase 1a

| | Daily | | | |
|---|---|---|---|---|
| | Dose | Number of Capsules per Strength (mg) Daily Dose | | |
| Cohort^{a} | (mg)^{b} | 1.0 | 2.0 | 7.0 |
| 1 | ≤21 | 0 | 0 | 3 |
| 2 | ≤42 | 0 | 0 | 6 |
| 3 | ≤77 | 0 | 0 | 11 |
| 4 | ≤100 | 0 | 0 | TBD |
| 5 | ≤150 | 0 | 0 | TBD |
| 6 | ≤200 | 0 | 0 | TBD |

^{a}De-escalation in dose, if required in response to DLT, is a mid-dose between the current dose and the previous lower dose with DLT subject incidence of <33%.
^{b}Dose level increases is 2-fold or less.

Dose escalation proceeds according to a modified 3+3 design. At each dose level, 3 to 6 subjects are initially exposed to compound A22. To account for early drop out, up to 6 subjects may be enrolled. If 0 of the 3 initial subjects treated at a given dose level experiences DLT, the dose is escalated, and 3 subjects are enrolled at the next higher dose level; and if more than 3 subjects are enrolled, all subjects are evaluated for DLT before escalation to the next dose level cohort. If 1 of the 3 initial subjects treated at a given dose level experiences DLT, then 3 additional subjects are enrolled at the same dose level (for a total of 6 subjects). If more than 3 subjects are enrolled, if 1 DLT is observed, the dose cohort are expanded to 6. If 0 of the additional subjects experiences DLT (i.e., DLT observed in 1 of the 6 subjects enrolled in the level), then the dose is escalated, and 3 subjects are enrolled at the next higher dose level. If 1 or more of the additional subjects experiences DLT (i.e., DLT observed in 2 or more of the 6 subjects enrolled in the level), it is concluded that the MTD has been exceeded. If 2 or more of the initial subjects treated at a given dose level experience DLT, then it is concluded that the MTD has been exceeded. If the MTD has been exceeded, then up to 6 subjects are enrolled at the next lower dose if fewer than 6 subjects were evaluated at the lower dose. The MTD is defined as the highest dose level of compound A22 at which no more than 1 of 6 subjects experiences DLT.

Each cycle is 28 days (4 weeks) and the DLT observation period is the first cycle (i.e., 28 days after initiation of dosing). A DLT is defined as a severe or clinically significant adverse event (AE) or abnormal laboratory value (Grade 3 or greater, unless otherwise specified), unless it is clearly related to disease progression, intercurrent illness, preexisting condition, or concomitant medications. Toxicity severity is graded according to the National Cancer Institute (NCI) Common Terminology Criteria for Adverse Events (CTCAE), Version 5.0. For purposes of dose escalation, the totality of accrued safety information across all cycles completed at the time of DEC data review is taken into consideration.

Barring DLT, sequential dose escalation of compound A22 has up to a total of 6 dose levels to a maximum of 200 mg; on the basis of these an MTD is identified. The MTD is defined as the highest dose level with a subject incidence of DLTs of 0 or 1 out of 6 during the first 28 days of the administration of compound A22. A minimum of 6 subjects are treated at a dose level before this dose level is deemed as the MTD. Subjects who receive less than 75% of planned doses due to reasons other than a DLT are replaced for the determination of the MTD, but remain part of the safety population if they receive at least one dose of compound A22. At dose levels equal to or below the MTD, up to 6 additional subjects per dose level cohort may be enrolled to obtain additional safety, PK, and pharmacodynamic (PD) data. The RP2D is determined based on a comprehensive analysis of safety, tolerability, PK/PD, and preliminary efficacy from the dose escalation phase.

Phase 1b is a cohort expansion phase. Once the RP2D is determined, up to 40 additional subjects are enrolled to evaluate safety and efficacy of compound A22 in subjects with a documented MYC genomic amplified/overexpressed tumor. Dosing in this phase of the study has the first cycle of therapy (i.e., 28 days). The DEC review cumulative safety and available PK data in subjects treated in Phase 1b for DLTs, with DEC reviews scheduled after the 6^{th}, 12^{th}, 24^{th}, 32^{nd}, and 40^{th} subjects complete a cycle of compound A22.

Subjects who complete one cycle of compound A22 in either Phase 1a or Phase 1b are offered continued access to compound A22 until disease progression or unacceptable toxicity. Dosing continues at the assigned dose or may be increased up to the dose determined to be tolerated in Study BTX-A51-001 (NCT04243785) or the current study. The DEC continues to review accruing safety/PK data, inclusive of all cycles, for subjects who continue with treatment.

Once treatment has completed, subjects are contacted by telephone every 3 months for survival status and anticancer therapy; the cause of death is documented. Individual subjects are considered to have completed the study 2 years after their last treatment or upon death, whichever occurs first. The study ends after a minimum of 70% of subjects have died or 5 years after the last subject is enrolled, whichever occurs first.

Up to approximately 112 subjects with an advanced solid tumor or NHL are enrolled. Phase 1a (dose escalation phase) enrolls up to 72 subjects, depending on the dose at which the MTD is determined; additional subjects may be enrolled in the event that a given subject either does not receive compound A22 or discontinues early for reasons other than safety and is not evaluable for toxicity. Phase 1b (cohort expansion phase) includes a basket cohort of up to approximately 40 subjects with an advanced malignancy and documented MYC genomic amplification/overexpression to further characterize the safety, tolerability, and preliminary efficacy when administered at the RP2D determined in Phase 1a. A sample size of 40 subjects treated at Phase 1b provides a high probability, approximately 92%, 99% or 100%, of detecting an AE with an incidence of 5%, 10% or 15% respectively.

Eligible subjects for the study are ≥18 years of age with a histologically or cytologically documented, incurable or metastatic solid tumor or B-cell NHL that is refractory to or intolerant of all standard therapy or for which no standard therapy is available. For Phase 1b, each eligible subject has documentation of MYC genomic amplification/overexpression by tumor or blood based analysis. Each eligible subject must have a measurable disease per Response Evaluation Criteria in Solid Tumors Version 1.1 (RECIST v1.1). For NHL, each eligible subject must have bi-dimensionally measurable disease on cross sectional imaging by computed tomography (CT) or magnetic resonance imaging (MRI) as defined by Lugano criteria. Cheson et al., J. Clin. Oncol. 2014, 32, 3059-68.

Additional inclusion criteria for the eligible subjects include (i) absolute neutrophil count (ANC) ≥1000 cells/μL; (ii) white blood cell (WBC) count >1500/μL; (iii) platelet count ≥100,000/μL; (iv) hemoglobin ≥9.0 g/dL; (v) serum AST and serum ALT of ≤3.0×upper limit of normal (ULN) (≤5.0×ULN in subjects with documented liver involvement); (vi) alkaline phosphatase ≤2.5×ULN (≤5.0×ULN in subjects with documented liver involvement or bone metastases); (vii) total serum bilirubin ≤2×ULN (except for subjects with documented Gilbert's syndrome); (viii) serum creatinine ≤2×ULN or creatinine clearance of ≥30 mL/min; and (ix) International normalized ratio (INR) and activated partial thromboplastin time (aPTT) ≤1.5×ULN.

The study excludes those with (i) life expectancy <3 months as determined by an investigator; (ii) treatment with any local or systemic antineoplastic therapy (including chemotherapy, hormonal therapy, or radiation) within 3 weeks prior to first dose of compound A22, except (a) hormonal therapy with gonadotropin-releasing hormone (GnRH) agonists or antagonists for prostate cancer, (b) hormone-replacement therapy or oral contraceptives, (c) herbal therapy intended as anti-cancer ≥1 week prior to first dose of compound A22, and palliative radiotherapy for bone metastases ≥2 weeks prior to first dose of compound A22; (iii) chronic use of corticosteroids in excess of 10 mg daily of prednisone or equivalent within 4 weeks prior to first dose of compound A22, except replacement doses of corticosteroids, e.g., prednisone 5-7.5 mg daily; (iv) major trauma or major surgery within 4 weeks prior to first dose of compound A22; (v) adverse events from prior anti-cancer therapy that have not resolved to Grade ≤1 except for alopecia or Grade ≤2 immunotherapy-related thyroid toxicity; (vi) history of, or known, central nervous system (CNS) disease involvement, or prior history of NCI CTCAE Grade ≥3 drug-related CNS toxicity; (vii) a clinically significant cardiac disease; (viii) second primary malignancy that has not been in remission for greater than 3 years, except non-melanoma skin cancer, cervical carcinoma in situ on biopsy or squamous intraepithelial lesion on Papanicolaou (PAP) smear, localized prostate cancer (Gleason score <6), or resected melanoma in situ; other localized, solid tumors in situ or other low risk cancers; and (ix) any serious underlying medical (e.g., pulmonary, renal, hepatic, gastrointestinal, or neurological) or psychiatric condition (e.g., alcohol or drug abuse, dementia or altered mental status) or any issue that would limit compliance with study requirements, impair the ability of the subject to understand informed consent, or that in the opinion of the investigator would contraindicate the subject's participation in the study or confound the results of the study.

Screening commences with obtaining the subject's signed informed consent and occurs up to 28 days prior to the first dosing of the study drug. Screening procedures include the following: medical history review; physical exam; vital signs; echocardiogram/MUGA scan, 12 lead electrocardiogram (ECG); ECOG performance status; prior/concomitant medication review; blood collection for pregnancy test (females of child bearing potential); chemistry, hematology, and coagulation; AE assessment; archival or recent biopsy formalin-fixed paraffin-embedded (FFPE) tissue block collection; and CT/MRI and positron emission tomography-computed tomography (PET-CT) in subjects with fluorodeoxyglucose [FDG]-avid NHL (scans that meet protocol requirements that are obtained as part of standard medical practice up to 6 weeks prior to Cycle 1 Day 1 are acceptable). Baseline tumor lesions are measured and characterized prior to Cycle 1 Day 1 to assess the subject disease status prior to beginning treatment.

Compound A22 as a di-p-toluenesulfonate salt is provided as orally administered, immediate-release capsules of 1.0 mg, 2.0 mg, and 7.0 mg each. Subjects who meet eligibility criteria receive compound A22 orally once daily on a weekly schedule of 5 days on/2 days off. Each cycle is 4 weeks (28 days). Safety and efficacy assessments occurs on an outpatient basis. In addition, subjects undergo post-baseline CT/MRI or PET-CT (as appropriate for FDG-avid lymphomas) scans for tumor response assessment. A bone marrow biopsy may be performed at the discretion of the investigator for lymphoma subjects as indicated to establish staging. For a given subject, one or more doses may be held as needed to manage toxicity. In some cases, the dose and/or schedule may be lowered in subsequently enrolled subjects in response to toxicity. A subject who does not show evidence of disease progression by clinical assessment or by CT/MRI or applicable scan may continue receiving study treatment until disease progression (clinical or radiographic), unacceptable toxicity, or withdrawal of consent.

An End of Treatment (EOT) Visit is conducted within 14 to 28 days after the last dose of compound A22 is administered, regardless of the reason for discontinuation. In addition, a Safety Follow-Up is done by phone 30 days after last dose of the study drug. Adverse events ≥Grade 2 ongoing at the 30-day Safety Follow-Up are followed until the event resolves to ≤Grade 1, stabilizes, the subject starts alternate therapy, returns to a status that is clinically acceptable in the judgment of the investigator, is lost to follow-up, or terminates with the subject's death.

The safety of compound A22 is evaluated by (i) adverse events (NCI CTCAE Version 5.0); (ii) clinical laboratory testing (hematology, chemistry, and coagulation); (iii) physical examinations; (iv) vital signs (blood pressure, pulse, respiratory rate, body temperature, and weight); (v) 12 lead ECGs; and (vi) evaluation of left ventricular ejection fraction (LVEF). Dose limiting toxicities are evaluated for determination of the MTD and/or RP2D. Throughout the study, safety is evaluated by a Dose Escalation Committee (DEC) that includes principal investigator(s), the sponsor's physician (in consultation with the sponsor's pharmacologist/pharmacokineticist as needed), and/or independent experts. The DEC reviews all cumulative available data and authorizes plans for the dosing of each subsequent cohort.

The preliminary efficacy of compound A22 is evaluated by (i) objective response rate (ORR; complete remission (CR)+partial remission (PR)), per an investigator's assessment (for a solid tumor, responses are evaluated using Response Evaluation Criteria in Solid Tumors Version 1.1 (RECIST v1.1); and for subjects with NHL, Lugano response criteria are used); (ii) best response (CR, PR, stable disease or progression); (iii) disease control rate (DCR; CR+PR+stable disease); (iv) duration of response (DOR); (v) progression-free survival (PFS), defined as the time from first enrollment into the study to the earlier of the first documentation of definitive disease progression or death due to any cause (summarized descriptively using the Kaplan Meier method); and (vi) overall survival (OS), defined as the time from first enrollment into the study to death due to any cause (summarized descriptively using the Kaplan Meier method).

The PK parameters of compound A22 are determined, including (i) maximum observed plasma concentration (Cₘₐₓ); (ii) observed time of peak concentration (Tₘₐₓ); (iii) overall exposure (area under the plasma concentration curve, AUC); and (iv) elimination half-life.

Additional peripheral blood samples at dose levels that may be associated with efficacy and/or selected toxicity are collected and stored for analysis for possible exploratory association with response or biomarker analyses, including, but not limited to. gene sequencing and gene expression profiling. Cytogenetics and mutation panel include (i) gene expression levels of target SE genes (i.e., Mcl1, MYC, MYB, and MDM2) by digital droplet polymerase chain reaction (PCR); (ii) MCL1, MYC, MDM2, and p53 protein expression levels; and/or (iii) gene mutation analysis by next generation sequencing.

Blood samples are obtained for biomarker evaluation from all eligible subjects. Archival tumor tissue samples obtained outside of this study for other purposes are collected from all subjects and used for biomarker evaluation.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A method of treating, preventing, or ameliorating one or more symptoms of a malignant solid tumor in a subject, comprising administering to the subject in need thereof a therapeutically effective amount of a compound of Formula (I): 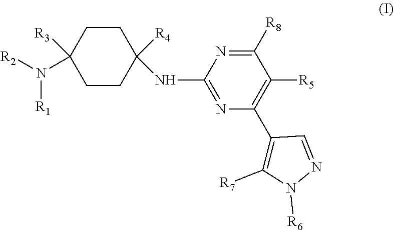
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein:
R₁, R₂, R₃, and R₄ are (a) or (b):
(a) R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or
R₁ and R₂ together with the nitrogen atom to which they are attached form heteroaryl or heterocyclyl; and
R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or
R₃ and R₄ are linked together to form C₁₋₆ alkylene;
(b) R₁ is (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, C(NR^{1a})NR^{1b}R^{1c}, C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R₂ together with R₃ and the carbon and nitrogen atoms to which they are attached form heterocyclylene; and
R₄ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₅, R₇, and R₈ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R₆ is hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, alkenyl, alkynyl, cycloalkyl, aryl, aralkyl, heteroaryl, heterocyclyl, and heterocyclylene is optionally substituted with one or more, in one embodiment, one, two, three, or four, substituents Q, wherein each Q is independently selected from: (a) deuterium, cyano, halo, imino, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR⁴, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) RD and Re together with the N atom to which they are attached form heterocyclyl, optionally substituted with one or more, in one embodiment, one, two, three, or four, substituents Q^{a};
wherein each Q^{a} is independently selected from: (a) deuterium, cyano, halo, nitro, imino, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The method of claim 1, wherein:
R₁ and R₂ are each independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; or
R₁ and R₂ together with the nitrogen atom to which they are attached form heteroaryl or heterocyclyl; and
R₃ and R₄ are each independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or
R₃ and R₄ are linked together to form C₁₋₆ alkylene;
wherein each alkyl, alkylene, alkenyl, alkynyl, cycloalkyl, aryl, aralkyl, heteroaryl, and heterocyclyl is optionally substituted with one or more substituents Q.

3. The method of claim 1 or 2, wherein R₁ is hydrogen or C₁₋₆ alkyl optionally substituted with one or more substituents Q.

4. The method of any one of claims 1 to 3, wherein R₁ is hydrogen, methyl, 1-pentyl, 2,2,2-trifluoroethyl, or pyrazol-4-ylmethyl.

5. The method of any one of claims 1 to 4, wherein R₁ is hydrogen.

6. The method of any one of claims 1 to 5, wherein R₂ is (i) hydrogen; (ii) C₁₋₆ alkyl, C₂₋₆ alkynyl, C₃₋₁₀ cycloalkyl, C₆₋₁₄ aryl, or C₇₋₁₅ aralkyl, each of which is optionally substituted with one or more substituents Q; or (iii) —C(O)R^{1a}.

7. The method of any one of claims 1 to 6, wherein R₂ is hydrogen, methyl, 1-pentyl, 2,2,2-trifluoroethyl, hydroxyl-methyl, 2-methoxyethyl, 3-aminopropyl, pyrazol-3-ylmethyl, pyrazol-4-ylmethyl, (1-methyl-pyrazol-4-yl)methyl, (3-methylpyrazol-4-yl)methyl, 1-(pyrazol-4-yl)ethyl, pyridin-3-yl-methyl, pent-4-yn-1-yl, 4-aminobicyclo[2.2.1]heptan-1-yl, 4-aminobicyclo[2.2.2]octan-1-yl, phenyl, benzyl, 2-methoxyacetyl, pent-4-ynoyl, pyrazol-3-ylcarbonyl, 3-cyclooctyloxy-carbonylaminopropyl, (E)-3-(cyclooct-4-en-1-yloxycarbonyl-amino)propyl, or (Z)-3-(cyclo-oct-4-en-1-yloxycarbonylamino)propyl.

8. The method of any one of claims 1 to 7, wherein R₂ is hydrogen.

9. The method of claim 1 or 2, wherein R₁ and R₂ together with the nitrogen atom to which they are attached form heteroaryl or heterocyclyl optionally substituted with one or more substituents Q.

10. The method of claim 1, 2, or 9, wherein R₁ and R₂ together with the nitrogen atom to which they are attached form pyrazol-1-yl, imidazol-1-yl, azetidin-1-yl, pyrrolidin-1-yl, piperidin-1-yl, or morpholin-4-yl, each of which is optionally substituted with one or more substituents Q.

11. The method of any one of claims 1 to 10, wherein R₃ is hydrogen or C₁₋₆ alkyl optionally substituted with one or more substituents Q.

12. The method of any one of claims 1 to 11, wherein R₃ is hydrogen or methyl.

13. The method of any one of claims 1 to 12, wherein R₃ is hydrogen.

14. The method of any one of claims 1 to 13, wherein R₄ is hydrogen or C₁₋₆ alkyl optionally substituted with one or more substituents Q.

15. The method of any one of claims 1 to 14, wherein R₄ is hydrogen or methyl.

16. The method of any one of claims 1 to 15, wherein R₄ is hydrogen.

17. The method of any one of claims 1 to 10, wherein R₃ and R₄ are linked together to form C₁₋₆ alkylene optionally substituted with one or more substituents Q.

18. The method of any one of claims 1 to 10 and 17, wherein R₃ and R₄ are linked together to form methanediyl or ethane-1,2-diyl.

19. The method of claim 1, wherein:
R₁ is (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, —C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R₂ together with R₃ and the carbon and nitrogen atoms to which they are attached form heterocyclylene; and
R₄ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(O)SR^{1a}, —C(NR^{1a})NR^{1b}R^{1c}, —C(S)R^{1a}, C(S)OR^{1a}, —C(S)NR^{1b}R^{1c}, —OR^{1a}, —OC(O)R^{1a}, —OC(O)OR^{1a}, —OC(O)NR^{1b}R^{1c}, —OC(O)SR^{1a}, —OC(NR^{1a})NR^{1b}R^{1c}, —OC(S)R^{1a}, —OC(S)OR^{1a}, —OC(S)NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(O)SR^{1d}, —NR^{1a}C(NR^{1d})NR^{1b}R^{1c}, —NR^{1a}C(S)R^{1d}, —NR^{1a}C(S)OR^{1d}, —NR^{1a}C(S)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
wherein each alkyl, alkenyl, alkynyl, cycloalkyl, aryl, aralkyl, heteroaryl, heterocyclyl, and heterocyclylene is optionally substituted with one or more substituents Q.

20. The method of claim 19, wherein R₁ is hydrogen or C₁₋₆ alkyl optionally substituted with one or more substituents Q.

21. The method of claim 19 or 20, wherein R₁ is hydrogen, methyl, 1-pentyl, 2,2,2-trifluoroethyl, or pyrazol-4-ylmethyl.

22. The method of any one of claims 1 and 19 to 21, wherein R₁ is hydrogen.

23. The method of any one of claims 1 and 19 to 22, wherein R₂ and R₃ together with the carbon and nitrogen atoms to which they are attached form heterocyclylene optionally substituted with one or more substituents Q.

24. The method of any one of claims 1 and 19 to 23, wherein R₂ and R₃ together with the carbon and nitrogen atoms to which they are attached form 5-oxo-pyrrolidin-2,2-diyl, 2,5-dioxo-imidazolidin-4,4-diyl, or 2-oxo-oxazolidin-4,4-diyl.

25. The method of any one of claims 1 and 19 to 24, wherein R₄ is hydrogen or C₁₋₆ alkyl optionally substituted with one or more substituents Q.

26. The method of any one of claims 1 and 19 to 25, wherein R₄ is hydrogen or methyl.

27. The method of any one of claims 1 and 19 to 26, wherein R₄ is hydrogen.

28. The method of any one of claims 1 to 27, wherein R₅ is hydrogen, deuterium, cyano, halo, nitro, or C₁₋₆ alkyl optionally substituted with one or more substituents Q.

29. The method of any one of claims 1 to 28, wherein R₅ is hydrogen, halo, or C₁₋₆ alkyl optionally substituted with one or more substituents Q.

30. The method of any one of claims 1 to 29, wherein R₅ is hydrogen, fluoro, chloro, or methyl.

31. The method of any one of claims 1 to 30, wherein R₅ is chloro.

32. The method of any one of claims 1 to 31, wherein R₆ is (i) hydrogen; or (ii) C₁₋₆ alkyl, C₃₋₁₀ cycloalkyl, or heterocyclyl, each of which is optionally substituted with one or more substituents Q.

33. The method of any one of claims 1 to 32, wherein R₆ is hydrogen, methyl, isopropyl, cyclopentyl, oxetan-3-yl, tetrafuran-3-yl, tetrahydropyran-3-yl, or tetrahydropyran-4-yl.

34. The method of any one of claims 1 to 33, wherein R₆ is methyl.

35. The method of any one of claims 1 to 33, wherein R₆ is hydrogen.

36. The method of any one of claims 1 to 35, wherein R₇ is C₁₋₆ alkyl optionally substituents with one or more substituents Q.

37. The method of any one of claims 1 to 36, wherein R₇ is C₁₋₆ alkyl substituted with C₃₋₁₀ cycloalkyl, wherein the alkyl and cycloalkyl are each optionally substituents with one or more substituents Q^{a}.

38. The method of any one of claims 1 to 37, wherein R₇ is tert-butyl, cyclopropylmethyl, 1-methylcyclopropylmethyl, 1-hydroxyl-cyclopropylmethyl, cyclobutylmethyl, or cyclopentylmethyl.

39. The method of any one of claims 1 to 38, wherein R₇ is cyclopropylmethyl, optionally substituents with one or more substituents Q.

40. The method of any one of claims 1 to 39, wherein R₈ is hydrogen.

41. The method of claim 1, wherein the compound is:
(1r,4r)-N¹-(4-(5-(cyclopropylmethyl)-1-methyl-1H-pyrazol-4-yl)pyrimidin-2-yl)cyclohexane-1,4-diamine A1;
N-((1r,4r)-4-((4-(5-(cyclopropylmethyl)-1-methyl-1H-pyrazol-4-yl)pyrimidin-2-yl)amino)cyclohexyl)-2-methoxyacetamide A2;
(1r,4r)-N¹-(4-(5-(cyclopropylmethyl)-1-methyl-1H-pyrazol-4-yl)pyrimidin-2-yl)-N⁴-methylcyclohexane-1,4-diamine A3;
(1r,4r)-N¹-(4-(5-(cyclopropylmethyl)-1-methyl-1H-pyrazol-4-yl)pyrimidin-2-yl)-N⁴,N⁴-dimethylcyclohexane-1,4-diamine A4;
(1r,4r)-N¹-(4-(1-cyclopentyl-5-(cyclopropylmethyl)-1H-pyrazol-4-yl)pyrimidin-2-yl)cyclohexane-1,4-diamine A5;
(1r,4r)-N¹-(4-(5-(cyclopropylmethyl)-1-(tetrahydro-2H-pyran-4-yl)-1H-pyrazol-4-yl)pyrimidin-2-yl)cyclohexane-1,4-diamine A6;
(1r,4r)-N¹-4-(5-(cyclopropylmethyl)-1-methyl-1H-pyrazol-4-yl)pyrimidin-2-yl)-N⁴-(2-methoxyethyl)cyclohexane-1,4-diamine A7;
8-((4-(5-(cyclopropylmethyl)-1-methyl-1H-pyrazol-4-yl)pyrimidin-2-yl)amino)-1,3-diazaspiro[4.5]decane-2,4-dione A8;
(1r,4r)-N¹-(4-(5-(cyclopropylmethyl)-1-isopropyl-1H-pyrazol-4-yl)pyrimidin-2-yl)cyclohexane-1,4-diamine A9;
(1r,4S)-N¹-(4-(5-(cyclopropylmethyl)-1-((S)-tetrahydrofuran-3-yl)-1H-pyrazol-4-yl)pyrimidin-2-yl)cyclohexane-1,4-diamine A10;
(1r,4S)-N¹-(4-(5-(cyclopropylmethyl)-1-((S)-tetrahydrofuran-3-yl)-1H-pyrazol-4-yl)pyrimidin-2-yl)cyclohexane-1,4-diamine A11;
(1r,4r)-N¹-(4-(5-(cyclopropylmethyl)-1-(oxetan-3-yl)-1H-pyrazol-4-yl)pyrimidin-2-yl)cyclohexane-1,4-diamine A12;
(1r,4r)-N¹-(4-(5-(cyclopentylmethyl)-1-methyl-1H-pyrazol-4-yl)pyrimidin-2-yl)cyclohexane-1,4-diamine A13;
(1r,4r)-N¹-(4-(5-(cyclopropylmethyl)-1-(tetrahydro-2H-pyran-3-yl)-1H-pyrazol-4-yl)pyrimidin-2-yl)cyclohexane-1,4-diamine A14;
(1r,4r)-N¹-4-(5-(cyclobutylmethyl)-1-methyl-1H-pyrazol-4-yl)pyrimidin-2-yl)cyclohexane-1,4-diamine A15;
(1-amino-4-((4-(5-(cyclopropylmethyl)-1-methyl-1H-pyrazol-4-yl)pyrimidin-2-yl)amino)cyclohexyl)methanol A16;
8-((4-(5-(cyclopropylmethyl)-1-methyl-1H-pyrazol-4-yl)pyrimidin-2-yl)amino)-3-oxa-1-azaspiro[4.5]decan-2-one A17;
4-(5-(cyclopropylmethyl)-1-methyl-1H-pyrazol-4-yl)-N-((1r,4r)-4-(piperidin-1-yl)cyclohexyl)pyrimidin-2-amine A18;
4-(5-(cyclopropylmethyl)-1-methyl-1H-pyrazol-4-yl)-N-((1r,4r)-4-morpholinocyclohexyl)-pyrimidin-2-amine A19;
4-(5-(cyclopropylmethyl)-1-methyl-1H-pyrazol-4-yl)-N-((1r,4r)-4-(pyrrolidin-1-yl)cyclohexyl)pyrimidin-2-amine A20;
N-((1r,4r)-4-(azetidin-1-yl)cyclohexyl)-4-(5-(cyclopropylmethyl)-1-methyl-1H-pyrazol-4-yl)pyrimidin-2-amine A21;
(1r,4r)-N¹-(5-chloro-4-(5-(cyclopropylmethyl)-1-methyl-1H-pyrazol-4-yl)pyrimidin-2-yl)cyclohexane-1,4-diamine A22;
(1r,4r)-N¹-(4-(5-(cyclopropylmethyl)-1-methyl-1H-pyrazol-4-yl)-5-methylpyrimidin-2-yl)cyclohexane-1,4-diamine A23;
(1r,4r)-N¹-(4-(5-(cyclobutylmethyl)-1-isopropyl-1H-pyrazol-4-yl)pyrimidin-2-yl)cyclohexane-1,4-diamine A24;
(4-(2-(((1r,4r)-4-aminocyclohexyl)amino)pyrimidin-4-yl)-1-methyl-1H-pyrazol-5-yl)(cyclopropyl)methanol A25;
(1r,4r)-N¹-(5-chloro-4-(5-(cyclopropylmethyl)-1-isopropyl-1H-pyrazol-4-yl)pyrimidin-2-yl)cyclohexane-1,4-diamine A26;
(1r,4r)-N¹-4-(1-methyl-5-((1-methylcyclopropyl)methyl)-1H-pyrazol-4-yl)pyrimidin-2-yl)cyclohexane-1,4-diamine A27;
(1r,4r)-N¹-4-(1-methyl-5-neopentyl-1H-pyrazol-4-yl)pyrimidin-2-yl)cyclohexane-1,4-diamine A28;
(1r,4r)-N¹-(4-(5-(cyclopropylmethyl)-1-methyl-1H-pyrazol-4-yl)pyrimidin-2-yl)-4-methylcyclohexane-1,4-diamine A29;
(1s,4s)-N¹-(5-chloro-4-(5-(cyclopropylmethyl)-1-methyl-1H-pyrazol-4-yl)pyrimidin-2-yl)-4-methylcyclohexane-1,4-diamine A30;
(1r,4r)-N¹-(4-(5-(cyclopropylmethyl)-1-methyl-1H-pyrazol-4-yl)-5-(trifluoromethyl)-pyrimidin-2-yl)cyclohexane-1,4-diamine A31;
N-((1r,4r)-4-(1H-pyrazol-1-yl)cyclohexyl)-5-chloro-4-(5-(cyclopropylmethyl)-1-methyl-1H-pyrazol-4-yl)pyrimidin-2-amine A32;
N-((1r,4r)-4-(1H-imidazol-1-yl)cyclohexyl)-5-chloro-4-(5-(cyclopropylmethyl)-1-methyl-1H-pyrazol-4-yl)pyrimidin-2-amine A33;
(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A34;
(5r,8r)-8-((5-chloro-4-(5-(cyclopropylmethyl)-1-methyl-1H-pyrazol-4-yl)pyrimidin-2-yl)amino)-1-azaspiro[4.5]decan-2-one A35;
(1r,4r)-N¹-benzyl-N⁴-(5-chloro-4-(5-(cyclopropylmethyl)-1-methyl-1H-pyrazol-4-yl)pyrimidin-2-yl)cyclohexane-1,4-diamine A36;
(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A37;
(1r,4r)-N¹-(5-chloro-4-(5-(cyclopropylmethyl)-1-methyl-1H-pyrazol-4-yl)pyrimidin-2-yl)-N⁴-(pyridin-3-ylmethyl)cyclohexane-1,4-diamine A38;
(1r,4r)-N¹-((1H-pyrazol-4-yl)methyl)-N⁴-(4-(5-(cyclopropylmethyl)-1-methyl-1H-pyrazol-4-yl)pyrimidin-2-yl)cyclohexane-1,4-diamine A39;
(1r,4r)-N¹-(4-(5-(cyclopropylmethyl)-1-methyl-1H-pyrazol-4-yl)pyrimidin-2-yl)-N⁴-((1-methyl-1H-pyrazol-4-yl)methyl)cyclohexane-1,4-diamine A40;
(1r,4r)-N¹-((1H-pyrazol-5-yl)methyl)-N⁴-(5-chloro-4-(5-(cyclopropylmethyl)-1-methyl-1H-pyrazol-4-yl)pyrimidin-2-yl)cyclohexane-1,4-diamine A41;
(1r,4r)-N¹-(1-(1H-pyrazol-4-yl)ethyl)-N⁴-(5-chloro-4-(5-(cyclopropylmethyl)-1-methyl-1H-pyrazol-4-yl)pyrimidin-2-yl)cyclohexane-1,4-diamine A42;
(1r,4r)-N¹-(5-chloro-4-(5-(cyclopropylmethyl)-1-methyl-1H-pyrazol-4-yl)pyrimidin-2-yl)-N⁴-phenylcyclohexane-1,4-diamine A43;
(1r,4r)-N¹-(5-chloro-4-(5-(cyclopropylmethyl)-1-methyl-1H-pyrazol-4-yl)pyrimidin-2-yl)-N⁴-((5-methyl-1H-pyrazol-4-yl)methyl)cyclohexane-1,4-diamine A44;
(1r,4r)-N¹-(5-chloro-4-(5-(cyclopropylmethyl)-1-methyl-1H-pyrazol-4-yl)pyrimidin-2-yl)-N⁴-(2,2,2-trifluoroethyl)cyclohexane-1,4-diamine A45;
(1r,4r)-N¹-((1H-pyrazol-4-yl)methyl)-N⁴-(5-chloro-4-(5-(cyclopropylmethyl)-1-methyl-1H-pyrazol-4-yl)pyrimidin-2-yl)-N¹-(2,2,2-trifluoroethyl)cyclohexane-1,4-diamine A46;
(1r,4r)-N¹,N¹-bis((1H-pyrazol-4-yl)methyl)-N⁴-(5-chloro-4-(5-(cyclopropylmethyl)-1-methyl-1H-pyrazol-4-yl)pyrimidin-2-yl)cyclohexane-1,4-diamine A47;
(1r,4r)-N¹-(4-(5-(cyclopropylmethyl)-1-methyl-1H-pyrazol-4-yl)-5-fluoropyrimidin-2-yl)cyclohexane-1,4-diamine A48;
(1r,4r)-N¹-((1H-pyrazol-4-yl)methyl)-N⁴-(4-(5-(cyclopropylmethyl)-1-methyl-1H-pyrazol-4-yl)-5-fluoropyrimidin-2-yl)cyclohexane-1,4-diamine A49;
(1r,4r)-N¹-(5-chloro-4-(5-(cyclopropylmethyl)-1H-pyrazol-4-yl)pyrimidin-2-yl)cyclohexane-1,4-diamine A50;
(1r,4r)-N¹-(4-(5-(cyclopropylmethyl)-1-methyl-1H-pyrazol-4-yl)-5-fluoropyrimidin-2-yl)-N⁴-(pent-4-yn-1-yl)cyclohexane-1,4-diamine A51;
(1r,4r)-N¹-(4-(5-(cyclopropylmethyl)-1-methyl-1H-pyrazol-4-yl)-5-chloropyrimidin-2-yl)-N⁴-(pent-4-yn-1-yl)cyclohexane-1,4-diamine A52;
N-((1r,4r)-4-((4-(5-(cyclopropylmethyl)-1-methyl-1H-pyrazol-4-yl)-5-chloropyrimidin-2-yl)amino)cyclohexyl)pent-4-ynamide A53;
N-((1r,4r)-4-((5-chloro-4-(5-(cyclopropylmethyl)-1-methyl-1H-pyrazol-4-yl)pyrimidin-2-yl)amino)cyclohexyl)pentanamide A54;
N¹-(5-chloro-4-(5-(cyclopropylmethyl)-1-methyl-1H-pyrazol-4-yl)pyrimidin-2-yl)bicyclo[2.2.1]heptane-1,4-diamine A55;
N¹-(5-chloro-4-(5-(cyclopropylmethyl)-1-methyl-1H-pyrazol-4-yl)pyrimidin-2-yl)bicyclo[2.2.2]octane-1,4-diamine A56;
(1r,4r)-N¹-(5-chloro-4-(5-(cyclopropylmethyl)-1-methyl-1H-pyrazol-4-yl)pyrimidin-2-yl)-N⁴-pentylcyclohexane-1,4-diamine A57;
(1r,4r)-N¹-(5-chloro-4-(5-(cyclopropylmethyl)-1-methyl-1H-pyrazol-4-yl)pyrimidin-2-yl)-N⁴, N⁴-dipentylcyclohexane-1,4-diamine A58;
(1r,4r)-N¹-(3-aminopropyl)-N⁴-(5-chloro-4-(5-(cyclopropylmethyl)-1-methyl-1H-pyrazol-4-yl)pyrimidin-2-yl)cyclohexane-1,4-diamine A59;
(Z)-cyclooct-4-en-1-yl (3-(((1r,4r)-4-((5-chloro-4-(5-(cyclopropylmethyl)-1-methyl-1H-pyrazol-4-yl)pyrimidin-2-yl)amino)cyclohexyl)amino)propyl)carbamate A60;
cyclooctyl (3-(((1r,4r)-4-((5-chloro-4-(5-(cyclopropylmethyl)-1-methyl-1H-pyrazol-4-yl)pyrimidin-2-yl)amino)cyclohexyl)amino)propyl)carbamate A61; or
(E)-cyclooct-4-en-1-yl (3-(((1r,4r)-4-((5-chloro-4-(5-(cyclopropylmethyl)-1-methyl-1H-pyrazol-4-yl)pyrimidin-2-yl)amino)cyclohexyl)amino)propyl)carbamate A62;
or a tautomer, a mixture of two or more tautomers, or an isotopic variant thereof; or a pharmaceutically acceptable salt, solvate, hydrate, or prodrug thereof.

42. The method of claim 1, wherein the compound is (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a pharmaceutically acceptable salt, solvate, hydrate, or prodrug thereof.

43. The method of claim 42, wherein the compound is a pharmaceutically acceptable salt of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a solvate, or hydrate thereof.

44. The method of claim 42 or 43, wherein the compound is a p-toluenesulfonate salt of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a solvate, or hydrate thereof.

45. The method of any one of claims 42 to 43, wherein the compound is a di-p-toluenesulfonate salt of (1r,4r)-N¹-(5-chloro-4-(5-(cyclopropylmethyl)-1-methyl-1H-pyrazol-4-yl)pyrimidin-2-yl)cyclohexane-1,4-diamine A22, or a tautomer, a mixture of two or more tautomers, or an isotopic variant thereof; or a solvate, or hydrate thereof.

46. The method of claim 45, wherein the di-p-toluenesulfonate salt is crystalline.

47. The method of claim 45 or 46, wherein the crystalline di-p-toluenesulfonate salt has an X-ray powder diffractogram comprising peaks at two-theta angles (°) of approximately 5.5, 7.6, and 21.9.

48. The method of claim 1, wherein the compound is (1r,4r)-N¹-(5-chloro-4-(5-(cyclopropylmethyl)-1H-pyrazol-4-yl)pyrimidin-2-yl)cyclohexane-1,4-diamine A50, or a tautomer, a mixture of two or more tautomers, or an isotopic variant thereof; or a pharmaceutically acceptable salt, solvate, hydrate, or prodrug thereof.

49. The method of any one of claims 1 to 48, wherein the malignant solid tumor is an advanced malignant solid tumor.

50. The method of any one of claims 1 to 49, wherein the malignant solid tumor is unresectable.

51. The method of any one of claims 1 to 50, wherein the malignant solid tumor is refractory.

52. The method of any one of claims 1 to 51, wherein the malignant solid tumor is relapsed.

53. The method of any one of claims 1 to 52, wherein the malignant solid tumor is metastatic.

54. The method of any one of claims 1 to 53, wherein the malignant solid tumor harbors an MYC aberration.

55. The method of any one of claims 1 to 54, wherein the malignant solid tumor is stage II, III, or IV.

56. The method of any one of claims 1 to 55, wherein the malignant solid tumor is stage III or IV.

57. The method of any one of claims 1 to 48, wherein the malignant solid tumor is lymphoma.

58. The method of claim 57, wherein the lymphoma is non-Hodgkin's lymphoma.

59. The method of claim 57 or 58, wherein the lymphoma is B-cell non-Hodgkin's lymphoma.

60. The method of any one of claims 57 to 59, wherein the lymphoma is advanced lymphoma.

61. The method of any one of claims 57 to 60, wherein the lymphoma is unresectable.

62. The method of any one of claims 57 to 61, wherein the lymphoma is refractory.

63. The method of any one of claims 57 to 62, wherein the lymphoma is relapsed.

64. The method of any one of claims 57 to 63, wherein the lymphoma is metastatic.

65. The method of any one of claims 57 to 64, wherein the lymphoma harbors an MYC aberration.

66. The method of any one of claims 57 to 65, wherein the lymphoma is stage II, III, or IV.

67. The method of any one of claims 57 to 66, wherein the lymphoma is stage III or IV.

68. The method of any one of claims 1 to 67, wherein the subject has failed a prior therapy.

69. The method of any one of claims 1 to 68, wherein the subject is a human.

70. The method of any one of claims 1 to 69, wherein the compound is administered orally.

71. The method of any one of claims 1 to 70, wherein the compound is administered as a tablet or capsule.

72. The method of any one of claims 1 to 71, wherein the therapeutically effective amount is ranging from about 0.01 to about 10 mg/kg per day.

73. The method of any one of claims 1 to 72, wherein the therapeutically effective amount is ranging from about 1 to about 500 mg per day.

74. The method of any one of claims 1 to 73, wherein the compound is administered in a cycle.

75. The method of any one of claims 1 to 74, wherein one cycle is 28 days.

76. The method of any one of claims 1 to 75, wherein the compound is administered for 5 days per week.

77. The method of any one of claims 1 to 76, wherein the compound is administered on Days 1, 2, 3, 4, and 5 in a week.

78. The method of any one of claims 1 to 77, wherein the compound is administered in a 28-day cycle for 5 days per week for 3 weeks, followed by 1 week of rest.

79. The method of any one of claims 1 to 78, wherein the compound is administered in a 28-day cycle on Days 1, 2, 3, 4, and 5 per week for 3 weeks followed by 1 week of rest.
